(19) United States
(12) Patent Application Publication
(10) Pub. No.: US 2022/0357328 A1
(43) Pub. Date: Nov. 10, 2022

(54) SUPERIOR BIOMARKER SIGNATURE TO PREDICT THE RESPONSE OF A BREAST CANCER PATIENT TO CHEMOTHERAPY

(71) Applicant: OAKLABS GmbH, Henningsdorf (DE)

(72) Inventors: Florian BURGER, Schonwalde (DE); Martina SCHAD, Berlin (DE); Jim KALLARACKAL, Berlin (DE)

(21) Appl. No.: 17/641,064

(22) PCT Filed: Sep. 3, 2020
(86) PCT No.: PCT/EP2020/074618

§ 371 Date: Mar. 7, 2022

(30) Foreign Application Priority Data
Sep. 9, 2019 (EP) 19196222.4

(51) Int. Cl. G01N 33/574; A61K 31/337

(52) U.S. Cl. CPC G01N 33/57415; G01N 33/57484; A61K 31/337; G01N 2333/4703

(57) ABSTRACT

The present invention relates to methods for predicting the response of a breast cancer patient to a chemotherapy. The present invention further relates to a method of determining whether to treat a breast cancer patient with a chemotherapy. The present invention also relates to a kit for predicting the response of a breast cancer patient to a chemotherapy.

The present invention relates to methods for predicting the response of a breast cancer patient to a chemotherapy. The present invention further relates to a method of determining whether to treat a breast cancer patient with a chemotherapy. The present invention also relates to a kit for predicting the response of a breast cancer patient to a chemotherapy.

### BACKGROUND OF THE INVENTION

Powerful profiling technologies and major achievements in molecular targeted therapies have triggered great expectations regarding precision medicine. However, matching patients and treatments in an optimal manner remains a pipe dream. Prerequisite for an efficient precision medicine is the correct prediction of patients who will respond or not respond to a specific treatment. Current predictions mainly rely on generic biomarkers of low complexity which are used to subgroup patients of a specific indication. The biomarkers often emerge from the pathways related to the mode of action of the treatment. In breast cancer, common biomarkers are protein expression levels of estrogen receptor (ER), progesterone receptor (PR) as well as human epidermal growth factor (HER2), or mutations in the genes BRCA1 and BRCA2 [1,2,3,4].

Clinical trials often focus on targeted therapy for specific subgroups towards the ambition to treat each patient optimally.

The death ligand 1 (PD-L1) is a well-known example for a single molecule biomarker and its expression is associated with the response to PD-1/PD-L1 inhibitors [5]. Thus, the response rates in PD-L1 selected patients are higher com-pared to unselected patients, e.g. 45.2% to 20% in the case of pembrolizumab in non-small-cell lung cancer [6]. The increase of the response rate of treated patients is an impressive improvement. However, only 23% to 28% of patients with non-small-cell lung carcinoma (NSCLC) [7,6] have a high level of PD-L1 expression and are considered to be eligible for a treatment with pembrolizumab. Consequently, a large fraction of patients would not be treated due to their PD-L1 test result though they would benefit.

This example illustrates the limitations of a single molecule biomarker which is not able to capture the biological complexity of response.

The capabilities of omics technologies build the basis to overcome this problem by generating large amounts of molecular data to combine several molecules in a multivariable model. Though numerous biomarker signatures have been published to classify responses to therapeutic drugs, only few could be validated in independent studies [8,9].

While single biomarkers may be insufficient in accurate patient stratification, biomarker signatures are accompanied by other challenges like overfitting and lack of reproducibility.

Oncotype Dx, EndoPredict, PAM50 and BreastCancer Index are some of the rare examples where biomarker signatures provide sufficient evidence of clinical utility, but none of them is able to guide choices of specific treatment regimes [10].

A prospective selection of patients who are most likely to respond to a given treatment is highly anticipated. Efforts are being made to develop biomarker signatures specifically for single drugs to predict pathologic complete response (pCR) or progression free survival (PFS). Hatzis et al., for example, identified genomic predictors of response and survival following chemotherapy for invasive breast cancer [11]. The selected genes and the model have been obtained by utilizing common statistics and ML libraries.

The present inventors were able to improve the results of Hatzis et al. by using dedicated new concepts which evolved from well approved algorithms applied in disciplines outside the life sciences. They combined algorithms from AI, pattern recognition and ML to identify the smallest set of features that is capable to achieve the greatest possible accuracy in predicting independent data out of tens of thousands of features. In particular, the present inventors used the Hatzis discovery cohort and validation cohort of in total 508 patients as basis [11] to: develop a biomarker signature of minimal size using the Hatzis discovery cohort of 310 patients, significantly improve the accuracy in predicting pCR in the validation cohort of 198 patients, and cross-validate the biomarker signature using 112 patients of the Hatzis validation cohort of two independent clinical sites. This approach is essential in order to translate a biomarker signature to clinical application.

The present inventors developed a 3-genes biomarker signature to predict the response to taxane chemotherapy in invasive breast cancer. The signature was validated using the Hatzis et al. validation cohort of 198 patients. They achieved a significant improvement in predicting responders and non-responders (pCR vs. RD) with an area under the receiver operating characteristics curve of 74%. With a model of just 3 genes the response rate could almost be increased by 33% compared to the benchmark published by Hatzis et al.

### SUMMARY OF THE INVENTION

In a first aspect, the present invention relates to a method of predicting the response of a breast cancer patient to a chemotherapy

based on a combination of levels determined from at least two biomarkers in a biological sample of the breast cancer patient,
wherein the at least two biomarkers are selected from three groups, the at least two biomarkers belonging to different groups, wherein the three groups comprise a first group, a second group, and a third group, wherein
the first group comprises SCUBE2, CA12, and ANXA9,
the second group comprises ELF5, ROPN1, ROPN1B, SOX10, TMEM158, FAM171A1, and SFRP1, and
the third group comprises NFIB and SFRP1.

In a second aspect, the present invention relates to the use of a combination of levels determined from at least two biomarkers in a biological sample of a breast cancer patient for predicting the response of the breast cancer patient to a chemotherapy,

wherein the at least two biomarkers are selected from three groups, the at least two biomarkers belonging to different groups, wherein the three groups comprise a first group, a second group, and a third group, wherein
the first group comprises SCUBE2, CA12, and ANXA9,
the second group comprises ELF5, ROPN1, ROPN1B, SOX10, TMEM158, FAM171A1, and SFRP1, and
the third group comprises NFIB and SFRP1.

In a third aspect, the present invention relates to a method of determining whether to treat a breast cancer patient with a chemotherapy comprising the steps of:
(i) carrying out the method according to the first aspect to obtain patient specific data,
(ii) determining whether to treat the breast cancer patient with a chemotherapy based on comparing the patient specific data with at least one reference criterion, and
(iii) if the patient specific data meets the at least one reference criterion recommending treatment of the patient with a chemotherapy.

In a fourth aspect, the present invention relates to method of predicting the response of a breast cancer patient to a chemotherapy comprising the step of: determining the level of at least one biomarker selected from the group consisting of SCUBE2 and ELF5 in a biological sample of a breast cancer patient.

In a fifth aspect, the present invention relates to a kit for predicting the response of a breast cancer patient to a chemotherapy comprising means for determining the level of at least one biomarker selected from the group consisting of SCUBE2 and ELF5 in a biological sample of a breast cancer patient.

This summary of the invention does not describe all features of the invention.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kolbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

The term, “cancer”, as used herein, includes a disease characterized by aberrantly regulated cellular growth, proliferation, differentiation, adhesion, and/or migration. The term “cancer” also comprises cancer metastases.

The term “metastasis”, as used herein, refers to the spread of cancer cells from their original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor at the target site depends on angiogenesis. Tumor metastasis often occurs even after the removal of the primary tumor because tumor cells or components may remain and develop metastatic potential.

In the context of the present invention, the cancer is breast cancer.

The term “breast cancer”, as used herein, relates to a type of cancer originating from breast tissue, most commonly from the inner lining of milk ducts or the lobules that supply the ducts with milk. Cancers originating from ducts are known as ductal carcinomas, while those originating from lobules are known as lobular carcinomas. Occasionally, breast cancer presents as metastatic disease. Common sites of metastasis include bone, liver, lung and brain. Breast cancer occurs in humans and other mammals. While the overwhelming majority of human cases occur in women, male breast cancer can also occur. In one embodiment of the present invention, the breast cancer is primary breast cancer (also referred to as early breast cancer). Primary breast cancer is breast cancer that hasn't spread beyond the breast or the lymph nodes under the arm. Preferably, the breast cancer is an invasive breast cancer.

The term “tumor”, as used herein, refers to all neoplastic cell growth and proliferation whether malignant or benign, and all pre-cancerous and cancerous cells and tissues. The terms “tumor” and “cancer” may be used interchangeably herein. In one embodiment of the present invention, the tumor is a solid tumor. In the context of the present invention, the tumor is a breast tumor.

Several molecular subtypes of breast cancer/tumors are known to the skilled person. The term “molecular subtype of a tumor” (or “molecular subtype of a cancer”), as used herein, refers to subtypes of a tumor/cancer that are characterized by distinct molecular profiles, e.g. gene expression profiles. In one embodiment, the molecular subtype is HER2-negative. In one particular embodiment, the molecular subtype is HER2-negative and progesterone receptor (PR)-positive breast cancer. In one another particular embodiment, the molecular subtype is HER2-negative and progesterone receptor (PR)-negative breast cancer.

The term “(therapeutic) treatment”, in particular in connection with the treatment of breast cancer, as used herein, relates to any treatment which improves the health status and/or prolongs (increases) the lifespan of a patient. Said treatment may eliminate cancer, reduce the size or the number of tumors in a patient, arrest or slow the development of cancer in a patient, inhibit or slow the development of new cancer in a patient, decrease the frequency or severity of symptoms in a patient, and/or decrease recurrences in a patient who currently has or who previously has had cancer. In one embodiment, the term “(therapeutic) treatment” is meant to refer to one or more of surgical removal of the primary tumor, chemotherapy, hormonal therapy, radiation therapy, and immunotherapy/targeted therapy. The term “(therapeutic) treatment” also covers “adjuvant therapy” as well as “neoadjuvant therapy”.

The term “adjuvant therapy”, as used herein, refers to a treatment that is given in addition to the primary, main, or initial treatment. The surgeries and complex treatment regimens used in cancer therapy have led the term to be used mainly to describe adjuvant cancer treatments. An example of adjuvant therapy is the additional treatment (e.g. chemotherapy) usually given after surgery (post-surgically), where all detectable disease has been removed, but where there remains a statistical risk of relapse due to occult disease.

The term “neoadjuvant therapy”, as used herein, refers to a treatment given before the primary, main, or initial treatment (e.g. pre-surgical chemotherapy).

The term “breast cancer treatment”, as used herein, may include surgery, medications (anti-hormonal/endocrine therapy and chemotherapy), radiation, immunotherapy/targeted therapy as well as combinations of any of the foregoing.

The term “chemotherapy”, as used herein, is a type of cancer treatment that uses one or more anti-cancer drugs (chemotherapeutic agents) as part of a standardized chemotherapy regimen. Chemotherapy may be given with a curative intent (which almost always involves combinations of drugs), or it may aim to prolong life or to reduce symptoms (palliative chemotherapy). Chemotherapy comprises the administration of chemotherapeutic agents. Chemotherapeutic agents encompass cytostatic compounds and cytotoxic compounds. Traditional chemotherapeutic agents act by killing cells that divide rapidly, one of the main properties of most cancer cells.

The term “chemotherapeutic agent”, as used herein, includes, but is not limited to, taxanes, platinum compounds, nucleoside analogs, camptothecin analogs, anthracyclines, anthracycline analogs, etoposide, bleomycin, vinorelbine, cyclophosphamide, antimetabolites, anti-mitotics, and alkylating agents. According to the present invention a reference to a chemotherapeutic agent is to include any prodrug such as ester, salt or derivative such as a conjugate of said agent. Examples are conjugates of said agent with a carrier substance, e.g. protein-bound paclitaxel such as albumin-bound paclitaxel. Preferably, salts of said agent are pharmaceutically acceptable. Chemotherapeutic agents are often given in combinations, usually for 3 to 6 months. One of the most common treatments is cyclophosphamide plus doxorubicin (adriamycin; belonging to the group of anthracyclines and anthracycline analogs), known as AC. Sometimes, a taxane drug, such as docetaxel, is added, and the regime is then known as CAT; taxane attacks the microtubules in cancer cells. Thus, in one embodiment, the chemotherapy, e.g. neoadjuvant or adjuvant chemotherapy, comprises administration of a taxane. Another common treatment, which produces equivalent results, is cyclophosphamide, methotrexate, which is an antimetabolite, and fluorouracil, which is a nucleoside analog (CMF). Another standard chemotherapeutic treatment comprises fluorouracil, epirubicin and cyclophosphamide (FEC), which may be supplemented with a taxane, such as docetaxel, or with vinorelbine. The therapy of breast cancer preferably comprises the administration of a chemotherapeutic agent, e.g. a taxane. Taxanes are an established treatment regimen for both early and metastatic breast cancer. The taxanes in clinical use include paclitaxel (Taxol, Bristol-Myers Squibb) and docetaxel (Taxotere, Sanofi-Aventis). Paclitaxel is a natural product isolated from the bark of the Western yew tree (Taxus brevifolia) and docetaxel is a semisynthetic analog. Taxanes exert an anticancer affect by attacking the microtubules. The anthracycline may be doxorubicin (also known as Adriamycin), daunorubicin, idarubicin, or epirubicin. The anthracycline group of compounds has a planar anthraquinone chromophore that can intercalate between adjacent base pairs of DNA. The chromophore is linked to a daunosamine sugar moiety. Of the clinically used anthracyclines, doxorubicin and daunorubicin were originally isolated from bacterial species while idarubicin or epirubicin are semisynthetic derivatives [1^{R},2^{R},3^{R},4^{E}]. Doxorubicin (DOX) and daunorubicin (DAUN) have also been formulated into liposomal preparations; the pegylated versions of encapsulated DOX are termed Doxil, Caelyx or pegylated liposomal doxorubicin (PLD), and the non-pegylated versions are Myocet (NPLD; non-pegylated liposomal doxorubicin) and DaunoXome (liposomal daunorubicin) [5^{R}, 6^{R}].

The term “chemotherapy”, as used herein, encompasses “neoadjuvant chemotherapy” and “adjuvant chemotherapy”. Neoadjuvant chemotherapy is given before the primary, main, or initial treatment (pre-surgical chemotherapy). Adjuvant chemotherapy is given in addition to the primary, main, or initial treatment (post-surgical chemotherapy). In one embodiment of the present invention, the chemotherapy is a neoadjuvant chemotherapy. In one another embodiment of the present invention, the chemotherapy is an adjuvant chemotherapy.

The term “patient”, as used herein, refers to an individual known to be affected by cancer such as breast cancer. The term “patient” further refers to an individual for whom it is desired to know whether she or he will respond to therapy such as chemotherapy and/or is qualified to be treated, e.g. by chemotherapy. The patient will be classified as being a responder or non-responder of a therapy such as chemotherapy and/or as being treatable or non-treatable, e.g. by chemotherapy. A patient which is not treatable by chemotherapy may then be treated with an alternative therapy such as radiotherapy, surgery, anti-hormonal/endocrine therapy, immunotherapy/targeted therapy as well as combinations of any of the foregoing. The term patient encompasses a human or another mammal. Preferably, the patient is a human. More preferably, the patient is a female.

The term “(control) subject”, as used herein, refers to an individual known to be affected by cancer such as breast cancer and known to be a responder or non-responder of a specific therapeutic treatment such as chemotherapy. In other words, the (control) subject is an individual from which it is known that she or he responded or not responded to therapy such as chemotherapy.

The term (control) subject encompasses a human or another mammal. Preferably, the (control) subject is a human. More preferably, the (control) subject is a female.

The term “responder”, as used herein, includes individuals where the cancer/tumor is eradicated, reduced or improved (mixed responder or partial responder) by therapy, or simply stabilized such that the disease is not progressing. In responders where the cancer is stabilized then the period of stabilization is preferably such that the quality of life and/or patients life expectancy is increased (for example stable disease for more than 6 months) in comparison to an individual that does not receive a treatment. In the context of the present invention, the individual preferably shows pathological complete response (pCR).

The term “non-responder”, as used herein, includes individuals whose symptoms with regard to the cancer/tumor are not improved or stabilized by therapy. A non-responder is preferably an individual with a residual invasive disease.

The term “pathological complete response (pCR)” (also designated as “pathological complete remission (pCR)”), as used herein, generally refers to (i) the absence of residual invasive cancer based on hematoxylin and eosin evaluation of the complete resected breast specimen and all sampled regional lymph nodes, following completion of chemotherapy (i.e., ypT0/Tis ypN0 in the current AJCC staging system), or (ii) the absence of residual invasive and in situ cancer based on hematoxylin and eosin evaluation of the complete resected breast specimen and all sampled regional lymph nodes following completion of chemotherapy (i.e., ypT0 ypN0 in the current AJCC staging system).

The term “pathological partial response (pPR)”, as used herein, means that the tumor/cancer responds to the treatment to some extent, for example where said tumor/cancer is reduced by >0%, 10%, 20%, 30%, 40%, 50%, 60%, 70%, 80%, or 90% or more.

The term “predicting the response of a patient to therapy”, as used herein, means determining whether the patient will respond to therapy or not. In the context of the present invention, the patient is a breast cancer patient and the therapy is chemotherapy. The method to predict the response of a patient to chemotherapy may result in a probability statement. For example, the method to predict the response of a patient to chemotherapy may provide a probability that the patient will respond to the therapy or a value indicative for that probability. A method of predicting the probability of response of a patient to therapy does not necessarily imply a 100% predictive ability, but may indicate that patients with certain characteristics (e.g. specific biomarker levels indicative for a response) are more likely to experience a favorable clinical response such as a pathological complete response (pCR) to the therapy than subjects who lack such characteristics. The probability that the patient will respond may be derived from the certain characteristics of the patient, e.g. the specific biomarker levels. However, as will be apparent to one skilled in the art, some individuals identified as more likely to experience a favorable clinical response may nonetheless fail to demonstrate measurable clinical response to the treatment. Similarly, some individuals predicted as non-responders may nonetheless exhibit a favorable clinical response to the treatment.

In particular, cut-offs (also referred to as “thresholds” herein) may be provided based on which a breast cancer patient can be predicted as responder or non-responder to therapy or classified as having a high probability of response (e.g. pCR) or low probability of response (e.g. non-pCR) upon breast cancer treatment. If, for example, a specific cut-off/threshold is met, the probability is high that the patient will respond to therapy. Conversely, if a specific cut-off/threshold is not met, the probability is low that the patient will respond to therapy. Pre-defined cut-offs/thresholds indicative for a responder or non-responder to therapy or for low probability of response (e.g. non-pCR) or high probability of response (e.g. pCR) can be readily determined by the skilled person based on her or his general knowledge and the technical guidance provided herein (see examples). The same applies to cut-offs/thresholds which may be used to determine whether to treat a breast cancer patient with a therapy such as chemotherapy or not. For example, concordance studies in a training setting can be used for the definition and validation of suitable thresholds/cut-offs. In one embodiment, the thresholds/cut-offs are defined based on one or more previous clinical studies or clinical data. Moreover, additional clinical studies or data acquisition may be conducted for the establishment and validation of the thresholds/cut-offs. The thresholds/cut-offs may be determined/defined by techniques known in the art. In one embodiment, the thresholds/cut-offs are determined/defined on the basis of the data for response (e.g. pCR) in training cohorts and/or validation cohorts by partitioning tests, ROC analyses or other statistical methods and are, preferably, dependent on a specific clinical utility.

A cut-off/threshold may be established by plotting a measure of the expression level of the relevant gene or the expression levels of the relevant genes for each patient. Generally, the responders and non-responders will be clustered about different axes/focal points. A cut-off/threshold may be established in the gap between the clusters by classical statistical methods or simply plotting a “best fit line” to establish a boundary between the two groups. Values, for example, above the pre-defined threshold, can be designated as values of responders and values, for example, below the pre-defined threshold can be designated as values of non-responders.

In addition, values, for example, above the predefined threshold can be designated as values where a specific treatment such as chemotherapy is recommended and values, for example, below the predefined threshold can be designated as values where a specific treatment such as chemotherapy is not recommended.

Optionally the characterization of the patient as a responder or non-responder can be performed by reference to a reference level. The standard may be a profile of at least one (control) subject from whom it is known to be a responder or non-responder or alternatively may be a numerical value. Such pre-determined standards may be provided in any suitable form, such as a printed list or diagram, computer software program, or other media.

The term “biological sample”, as used herein, refers to any biological sample from a patient or (control) subject comprising at least one of the biomarkers referred to herein. The biological sample may be a body fluid sample, e.g. a blood sample or urine sample, or a tissue sample. Said biological sample may be provided by removing a body fluid from a patient or (control) subject, but may also be provided by using a previously isolated sample. For example, a blood sample may be taken from patient or (control) subject by conventional blood collection techniques. The biological sample, e.g. urine sample or blood sample, may be obtained from a patient or (control) subject prior to the initiation of a therapeutic treatment, during the therapeutic treatment, and/or after the therapeutic treatment. If the biological sample is obtained from at least one (control) subject, e.g. from at least 1, 2, 3, 4, 5, 6, 7, 8, 9, 10, 20, 30, 40, 50, 60, 70, 80, 90, 100, 150, 200, 250, 300, 400, 500, or 1.000 (control) subjects, it is designated as a “reference biological sample”. Preferably, the reference biological sample is from the same source as the biological sample of the patient to be tested. It is further preferred that both are from the same species, e.g. from a human. It is also (alternatively or additionally) preferred that the measurements of the reference biological sample of the (control) subject and the biological sample of the patient to be tested are identical, e.g. both have an identical volume. It is particularly preferred that the reference biological sample and the biological sample are from (control) subjects and patients of the same sex and similar age, e.g. no more than 2 years apart from each other.

The term “body fluid sample”, as used herein, refers to any liquid sample derived from the body of a patient or (control) subject comprising at least one of the biomarkers referred to herein. Said body fluid sample may be a urine sample, blood sample, sputum sample, breast milk sample, cerebrospinal fluid (CSF) sample, cerumen (earwax) sample, gastric juice sample, mucus sample, lymph sample, endolymph fluid sample, perilymph fluid sample, peritoneal fluid sample, pleural fluid sample, saliva sample, sebum (skin oil) sample, semen sample, sweat sample, tears sample, cheek swab, vaginal secretion sample, liquid biopsy, or vomit sample including components or fractions thereof.
The term “blood sample”, as used herein, encompasses a whole blood sample or a blood fraction such as blood cells, serum, or plasma.
In one embodiment of the present invention, the term “breast tumor sample” refers to a breast tumor tissue sample isolated from the cancer patient (e.g. a biopsy or resection tissue of the breast tumor). The breast tumor tissue sample may be a cryo-section of a breast tumor tissue sample or may be a chemically fixed breast tumor tissue sample. For example, the breast tumor tissue sample may be a formalin-fixed and paraffin-embedded (FFPE) breast tumor tissue sample. The sample of the breast tumor may also be (total) RNA extracted from the breast tumor tissue sample. The sample of the breast tumor may further be (total) RNA extracted from a FFPE breast tumor tissue sample. The breast tumor sample may also be a sample of one or more circulating tumor cells (CTCs) or (total) RNA extracted from the one or more CTCs. Those skilled in the art are able to perform RNA extraction procedures. For example, total RNA from a 5 to 10 μm curl of FFPE tumor tissue can be extracted using the High Pure RNA Paraffin kit (Roche, Basel, Switzerland) or the XTRAKT RNA Extraction kit XL (Stratifyer Molecular Pathology, Cologne, Germany). It is also possible to store the sample material to be used/tested in a freezer and to carry out the methods of the present invention at an appropriate point in time after thawing the respective sample material. A “pre-treatment” breast tumor sample is obtained from the breast cancer patient prior to initiation/administration of breast cancer treatment.

According to the present invention, the term “RNA transcript” includes and preferably relates to “mRNA” which means “messenger RNA” and relates to a “transcript” which encodes a peptide or protein. mRNA typically comprises a 5′ non-translated region (5′-UTR), a protein or peptide coding region and a 3′ non-translated region (3′-UTR). mRNA has a limited halftime in cells and in vitro. It should be noted that the term “RNA transcript” encompasses any RNA transcript of the gene selected from the group consisting of SCUBE2, CA12, ANXA9, ELF5, ROPN1, ROPN1B, SOX10, TMEM158, FAM171A1, NFIB, and SFRP1. Thus, the determination of the level of the biomarker SCUBE2 encompasses the determination of the level of any RNA transcript of said biomarker, the determination of the level of the biomarker CA12 encompasses the determination of the level of any RNA transcript of said biomarker, the determination of the level of the biomarker ANXA9 encompasses the determination of the level of any RNA transcript of said biomarker, the determination of the level of the biomarker ELF5 encompasses the determination of the level of any RNA transcript of said biomarker, the determination of the level of the biomarker ROPN1 encompasses the determination of the level of any RNA transcript of said biomarker, the determination of the level of the biomarker ROPN1B encompasses the determination of the level of any RNA transcript of said biomarker, the determination of the level of the biomarker SOX10 encompasses the determination of the level of any RNA transcript of said biomarker, the determination of the level of the biomarker TMEM158 encompasses the determination of the level of any RNA transcript of said biomarker, the determination of the level of the biomarker FAM171A1 encompasses the determination of the level of any RNA transcript of said biomarker, the determination of the level of the biomarker NFIB encompasses the determination of the level of any RNA transcript of said biomarker, and the determination of the level of the biomarker SFRP1 encompasses the determination of the level of any RNA transcript of said biomarker.

The term “level of a biomarker” refers to an amount (measured for example in grams, mole, or ion counts) or concentration of said biomarker (e.g. of the genes SCUBE2, CA12, ANXA9, ELF5, ROPN1, ROPN1B, SOX10, TMEM158, FAM171A1, NFIB, or SFRP1). If more than one biomarker is measured, the level is a sum, median, average, or product of the individual levels of each biomarker added up. The term “level”, as used herein, also comprises scaled, normalized, or scaled and normalized values or amounts. Preferably, the level is an expression level.

The term “expression level”, as used herein, refers to the level of expression of a particular biomarker (e.g. of the genes SCUBE2, CA12, ANXA9, ELF5, ROPN1, ROPN1B, SOX10, TMEM158, FAM171A1, NFIB, or SFRP1) so as to produce a transcript and/or protein. According to the present invention, the expression level is preferably determined on the RNA transcript level, in particular mRNA level (transcriptional level), for example, by measuring the transcribed mRNA (e.g. via microarray or northern blot), by reverse transcription (RT) quantitative PCR or by directly staining the mRNA (e.g. via in situ hybridization). The expression level of mRNA may be determined by microarray (using probes) or reverse transcription quantitative PCR (RT-qPCR) (using primers). As RNA cannot be directly amplified in PCR, it must be reverse transcribed into cDNA using the enzyme reverse transcriptase. For this purpose, a one-step RT-qPCR can be utilized, which combines the reactions of reverse transcription with DNA amplification by PCR in the same reaction. In one-step RT-qPCR, the RNA template is mixed in a reaction mix containing reverse transcriptase, DNA polymerase, primers and probes, dNTPs, salts and detergents. In a first step, the target RNA is reverse transcribed by the enzyme reverse transcriptase using the target-specific reverse primers. Afterwards, the cDNA is amplified in a PCR reaction using the primers/probes and DNA polymerase.

The quantitative PCR may be fluorescence-based quantitative real-time PCR, in particular fluorescence-based quantitative real-time PCR. The fluorescence-based quantitative real-time PCR comprises the use of a fluorescently labeled probe. The fluorescently labeled probe may consist of an oligonucleotide labeled with both a fluorescent reporter dye and a quencher dye (=dual-label probe).
As mentioned above, the level of the RNA transcript of the biomarker is preferably determined. The level of the RNA transcript (e.g. of the genes SCUBE2, CA12, ANXA9, ELF5, ROPN1, ROPN1B, SOX10, TMEM158, FAM171A1, NFIB, or SFRP1) is preferably determined using polynucleotides being specific for the target RNA transcript, in particular target mRNA-sequence. Said polynucleotides may be target RNA transcript/target mRNA-sequence specific primers or probes.

The wording “specific for the target mRNA-sequence”, as used in connection with primers or probes for use in accordance with the present invention, is meant to refer to the ability of the primers or probes to hybridize (i.e. anneal) to the target sequence. In particular, the wording “specific for the target mRNA-sequence” refers to the ability of the primer to hybridize (i.e. anneal) to the cDNA of the target mRNA-sequence under appropriate conditions of temperature and solution ionic strength, in particular PCR conditions. The conditions of temperature and solution ionic strength determine the stringency of hybridization. Hybridization requires that the two nucleic acids (i.e. primer and cDNA) contain complementary sequences, although depending on the stringency of the hybridization, mismatches between bases are possible. In one embodiment, “appropriate conditions of temperature and solution ionic strength” refer to a temperature in the range of from 58° C. to 62° C. (preferably a temperature of approximately 60° C.) and a solution ionic strength commonly used in PCR reaction mixtures. In one embodiment, the sequence of the primer is 80%, preferably 85%, more preferably 90%, even more preferably 95%, 96%, 97%, 98%, 99% or 100% complementary to the corresponding sequence of the cDNA of the target mRNA-sequence, as determined by sequence comparison algorithms known in the art.

In one embodiment, the primers/probes hybridize to the target sequence under stringent or moderately stringent hybridization conditions. In one preferred embodiment, the primer hybridizes to the cDNA of the target mRNA-sequence under stringent or moderately stringent hybridization conditions. “Stringent hybridization conditions”, as defined herein, involve hybridizing at 68° C. in 5×SSC/5×Denhardt's solution/1,0% SDS, and washing in 0,2×SSC/0,1% SDS at room temperature, or involve the art-recognized equivalent thereof (e.g., conditions in which a hybridization is carried out at 60° C. in 2,5×SSC buffer, followed by several washing steps at 37° C. in a low buffer concentration, and remains stable). “Moderately stringent hybridization conditions”, as defined herein, involve including washing in 3×SSC at 42° C., or the art-recognized equivalent thereof. The parameters of salt concentration and temperature can be varied to achieve the optimal level of identity between the primer and the target nucleic acid. Guidance regarding such conditions is available in the art, for example, by J. Sambrook et al. eds., 2000, Molecular Cloning: A Laboratory Manual, 3^{rd} Edition, Cold Spring Harbor Laboratory Press, Cold Spring Harbor; and Ausubel et al. eds., 1995, Current Protocols in Molecular Biology, John Wiley and Sons, N.Y.
In one embodiment, the probe hybridizes to the (amplified) cDNA of the target mRNA-sequence under stringent or moderately stringent hybridization conditions as defined above. The probes as defined above are preferably labeled, e.g., with a label selected from a fluorescent label, a fluorescence quenching label, a luminescent label, a radioactive label, an enzymatic label and combinations thereof. Preferably, the probes as defined above are dual-label probes comprising a fluorescence reporter moiety and a fluorescence quencher moiety.

In the context of the present patent application, the level of the following biomarkers is determined/further analyzed, preferably on the RNA transcript level:

SCUBE2 (geneID=57758, human) and its correlated genes CA12 (geneID=771, human) and ANXA9 (geneID=8416, human),
ELF5 (geneID=2001, human) and its correlated genes ROPN1 (geneID=54763, human), ROPN1B (geneID=152015, human), SOX10 (geneID=6663, human), TMEM158 (geneID=25907, human), FAM171A1 (geneID=221061, human), and SFRP1 (geneID=6422, human), and
NFIB (geneID=4781, human) and its correlated gene SFRP1 (geneID=6422, human).

The geneIDs have been taken from the National Center for Biotechnology Information (NCBI).

The above-mentioned biomarkers are genes, preferably from humans.

Two genes are said to be correlated if their variation about their respective mean values is not statistically independent, but mutually and linearly related. The Pearson correlation coefficient, which normalizes the expectation value of the common variation about the mean value of the genes with the product of the standard deviations of the two gene's signals, has been used here.

SCUBE2 (Signal peptide-complement protein C1r/C1s, Uegf, and Bmp1 [CUB]-epidermal growth factor [EGF] domain-containing protein or Signal Peptide, CUB Domain And EGF Like Domain Containing 2) is a 807-amino acids protein that belongs to a small family of three members. SCUBE2 is predominantly expressed in vascular endothelial cells [17] and regulates the SHH (Sonic Hedgehog) signaling, acting upstream of ligand binding at the plasma membrane [18]. Mounting evidence suggests that SCUBE2 acts as a tumor suppressor in breast cancer [19,20], NSCLC [21], colorectal cancer [22] and gastric cancer [23].

ELF5 (E74 Like ETS Transcription Factor 5 or E74 Like E26 transformation-specific [ETS] Transcription Factor 5) is a 265-amino acids protein and a member of the ETS family of transcription factors. ETS family proteins regulate a wide spectrum of biological processes and several ETS factors have been implicated with cancer initiation, progression and metastasis [25,26]. For ELF5, both tumor promoting and suppressive roles have been reported in breast cancer [27].

NFIB (Nuclear Factor I B) belongs to the nuclear factor 1 (NFI) family of transcription factors which control expression of a large number of cellular genes [29,30]. In a hetero and homodimer complex, the four members of the NFI family can activate or repress transcription depending on the context[30]. NFIB has been defined as an oncogene in several reports [31,32]. The chromosomal region encoding NFIB is amplified in TNBC[33].

CA12 (Carbonic Anhydrase 12) belongs to the carbonic anhydrase family. This is a large family of zinc metalloenzymes that catalyze the reversible hydration of carbon dioxide. They participate in a variety of biological processes, including, respiration, calcification, acid-base balance, bone resorption, and the formation of aqueous humor, cerebrospinal fluid, saliva, and gastric acid.

ANXA9 (Annexin A9) belongs to the family of annexins. This family is a family of calcium-dependent phospholipid-binding proteins. Members of the annexin family contain 4 internal repeat domains, each of which includes a type II calcium-binding site. The calcium-binding sites are required for annexins, for example, to aggregate and cooperatively bind anionic phospholipids and extracellular matrix proteins.

The protein encoded by the ROPN1 (Rhophilin Associated Tail Protein 1) gene is found in cancer tissue.

ROPN1B (Rhophilin Associated Tail Protein 1B) is a protein coding gene. Gene Ontology (GO) annotations related to this gene include, for example, protein homodimerization activity and receptor signaling complex scaffold activity. An important paralog of this gene is ROPN1.

The gene SOX10 (SRY-Box transcription factor 10) encodes a member of the SOX (SRY-related HMG-box) family of transcription factors involved in the regulation of embryonic development and in the determination of the cell fate. The encoded protein may act as a transcriptional activator after forming a protein complex with other proteins. This protein acts as a nucleocytoplasmic shuttle protein and is important for neural crest and peripheral nervous system development.

Transcription of the gene TMEM158 (Transmembrane Protein 158) is, for example, upregulated in response to activation of the Ras pathway, but not under other conditions that induce senescence.

FAM171A1 (Family With Sequence Similarity 171 Member A1) is a protein encoding gene. It is, for example, involved in the regulation of the cytoskeletal dynamics, plays a role in actin stress fiber formation.

The gene SFRP1 (Secreted Frizzled Related Protein 1) encodes a member of the SFRP family that contains a cysteine-rich domain homologous to the putative Wnt-binding site of Frizzled proteins. Members of this family act, for example, as soluble modulators of Wnt signaling; epigenetic silencing of SFRP genes leads to deregulated activation of the Wnt-pathway which is associated with cancer.

The term “sensitivity”, as used herein, refers to the number of true positive patients (%) with regard to the number of all positive patients (100%), where “true” means that the label assigned to the patient by the classification result coincides with the patient's actual label (positive or negative). The sensitivity is calculated by the following formula: Sensitivity=TP/(P) (TP=true positives; P=positives).

The term “specificity”, as used herein, relates to the number of true negative patients (%) with regard to the number of all negative patients (100%). The specificity is calculated by the following formula: Specificity=TN/(N) (TN=true negatives; N=negatives).

The term “accuracy”, as used herein, means a statistical measure for the correctness of classification or identification of sample types. The accuracy is the proportion of true results (both true positives and true negatives). The accuracy is calculated by the following formula: Accuracy=(TP+TN)/(P+N).

The term “AUC”, as used herein, relates to an abbreviation for the area under a curve. In particular, it refers to the area under a Receiver Operating Characteristic (ROC) curve. The term “Receiver Operating Characteristic (ROC) curve”, as used herein, refers to a plot of the true positive rate against the false positive rate for the different possible cut points of a test. It shows the trade-off between sensitivity and specificity depending on the selected cut point (any increase in sensitivity will be accompanied by a decrease in specificity). The area under an ROC curve is a measure for the accuracy of a diagnostic test (the larger the area the better, optimum is 1, a random test would have a ROC curve lying on the diagonal with an area of 0.5 (see, for reference, for example, JP. Egan. Signal Detection Theory and ROC Analysis).

As used herein, the term “kit of parts (in short: kit)” refers to an article of manufacture comprising one or more containers and, optionally, a data carrier. Said one or more containers may be filled with one or more of the above mentioned means or reagents. Additional containers may be included in the kit that contain, e.g., diluents, buffers and further reagents such as dNTPs. Said data carrier may be a non-electronical data carrier, e.g., a graphical data carrier such as an information leaflet, an information sheet, a bar code or an access code, or an electronical/computer-readable data carrier such as a compact disk (CD), a digital versatile disk (DVD), a microchip or another semiconductor-based electronical data carrier. The access code may allow the access to a database, e.g., an internet database, a centralized, or a decentralized database. Said data carrier may comprise instructions for the use of the kit in the methods of the invention. The data carrier may comprise threshold values or reference levels of (relative) expression levels of mRNA or of the scores calculated according to the methods of the present invention. In case that the data carrier comprises an access code which allows the access to a database, said threshold values or reference levels are deposited in this database. In addition, the data carrier may comprise information or instructions on how to carry out the methods of the present invention.

### Embodiments of the Invention

Examples where biomarker signatures provide sufficient evidence of clinical utility are very limited. In addition, none of them is able to guide choices of specific treatment regimes. A prospective selection of patients who are most likely to respond to a given treatment would be, thus, highly appreciated. Efforts are being made to develop biomarker signatures specifically for single drugs to predict pathologic complete response (pCR) or progression free survival (PFS). Hatzis et al., for example, identified genomic predictors of response and survival following chemotherapy for invasive breast cancer [11].

The present inventors were able to improve the results of Hatzis et al. by using dedicated new concepts which evolved from well approved algorithms applied in disciplines outside the life sciences. They combined algorithms from AI, pattern recognition and ML to identify the smallest set of features that is capable to achieve the greatest possible accuracy in predicting independent data out of tens of thousands of features.
In particular, the present inventors developed a 3-genes biomarker signature to predict the response to chemotherapy in invasive breast cancer. The signature was validated using the Hatzis et al. validation cohort of 198 patients. They achieved a significant improvement in predicting responders and non-responders (pCR vs. RD) with an area under the receiver operating characteristics curve of 74%. With a model of just 3 genes the response rate could almost be increased by 33% compared to the benchmark published by Hatzis et al.

Thus, in a first aspect, the present invention relates to a (an in vitro) method of predicting the response of a breast cancer patient to a chemotherapy based on a combination of levels determined/obtained from at least two biomarkers in a biological sample of the breast cancer patient,

wherein the at least two biomarkers are selected from three groups, the at least two (e.g. 2, 3, 4, 5, 6, 7, 8, 9, 10, 11, or 12) biomarkers belonging to different groups, wherein the three groups comprise a first group, a second group, and a third group, wherein
the first group comprises SCUBE2, CA12, and ANXA9,
the second group comprises ELF5, ROPN1, ROPN1B, SOX10, TMEM158, FAM171A1, and SFRP1, and
the third group comprises NFIB and SFRP1.

Preferably, the at least two biomarkers belonging to different groups and differ from each other. This means that it is not possible to select SFRP1 from the second group and SFRP1 from the third group.

For example,

one biomarker is selected from the first group and another biomarker is selected from the second group,
one biomarker is selected from the first group and another biomarker is selected from the third group, or
one biomarker is selected from the second group and another biomarker is selected from the third group.

In one preferred embodiment,

the biomarker SCUBE2 is selected from the first group and the biomarker ELF5 is selected from the second group,
the biomarker SCUBE2 is selected from the first group and the biomarker NFIB is selected from the third group, or
the biomarker ELF5 is selected from the second group and the biomarker NFIB is selected from the third group.

It is also possible to select more than one biomarker from a single group under the proviso that at least two biomarkers from different groups are selected.

In one more preferred embodiment, the combination of levels is determined from at least three biomarkers, at least one first biomarker, at least one second biomarker, and at least one third biomarker, wherein

the at least one first biomarker is selected from the first group consisting of SCUBE2, CA12, and ANXA9,
the at least one second biomarker is selected from the second group consisting of ELF5, ROPN1, ROPN1B, SOX10, TMEM158, FAM171A1, and SFRP1, and the at least one third biomarker is selected from the third group consisting of NFIB and SFRP1.

In one even more preferred embodiment, the at least one first biomarker is SCUBE2, the at least one second biomarker is ELF5, and the at least one third biomarker is NFIB.

CA12 and ANXA9 are biomarkers which correlate with SCUBE2 and, thus, may be used in addition or alternatively to SCUBE2. ROPN1, ROPN1B, SOX10, TMEM158, FAM171A1, and SFRP1 are biomarkers which correlate with ELF5 and, thus, may be used in addition or alternatively to ELF5. SFRP1 is a biomarker which correlates with NFIB and, thus, may be used in addition or alternatively to NFIB.

Prior to the provision/calculation of the combination of levels, the method preferably comprises the step of determining the level of at least two biomarkers in a biological sample of the breast cancer patient,

wherein the at least two biomarkers are selected from three groups, the at least two (e.g. 2, 3, 4, 5, 6, 7, 8, 9, 10, 11, or 12) biomarkers belonging to different groups, wherein the three groups comprise a first group, a second group, and a third group, wherein
the first group comprises SCUBE2, CA12, and ANXA9,
the second group comprises ELF5, ROPN1, ROPN1B, SOX10, TMEM158, FAM171A1, and SFRP1, and
the third group comprises NFIB and SFRP1.

In addition, prior to the provision/calculation of the combination, the method more preferably comprises the step of determining the level of at least three biomarkers in a biological sample of the breast cancer patient, at least one first biomarker, at least one second biomarker, and at least one third biomarker, wherein

the at least one first biomarker is selected from the first group consisting of SCUBE2, CA12, and ANXA9,
the at least one second biomarker is selected from the second group consisting of ELF5, ROPN1, ROPN1B, SOX10, TMEM158, FAM171A1, and SFRP1, and
the at least one third biomarker is selected from the third group consisting of NFIB and SFRP1.

In one embodiment, the combination of levels determined from the at least two biomarkers, preferably from the at least three biomarkers, more preferably from the biomarkers SCUBE2, ELF5, and NFIB, comprises calculating a sum of the levels, where the sum has a plurality of summands and each summand of the plurality of summands is derived from one, preferably from only one, of the levels.

In one embodiment, the combination of levels determined from the at least two biomarkers, preferably from the at least three biomarkers, more preferably from the biomarkers SCUBE2, ELF5, and NFIB, comprises a linear combination of levels, wherein the levels in the linear combination are weighted differently. A linear combination may be the sum of the levels each level multiplied with an associated coefficient, where different levels, preferably, have different coefficients. The respective coefficient may be different from 1 and/or positive or negative. Each arbitrarily selected pair of coefficients may have different coefficients.

In one embodiment, the method comprises the step of calculating patient-specific data from the combination, preferably linear combination, of levels determined from the at least two biomarkers, preferably from the at least three biomarkers, more preferably from the biomarkers SCUBE2, ELF5, and NFIB. Thus, in one particular embodiment, the method of predicting the response of a breast cancer patient to a chemotherapy comprises the steps of:
(i) providing a combination of levels determined/obtained from at least two biomarkers in a biological sample of the breast cancer patient, wherein the at least two biomarkers are selected from three groups, the at least two (e.g. 2, 3, 4, 5, 6, 7, 8, 9, 10, 11, or 12) biomarkers belonging to different groups, wherein the three groups comprise a first group, a second group, and a third group, wherein the first group comprises SCUBE2, CA12, and ANXA9, the second group comprises ELF5, ROPN1, ROPN1B, SOX10, TMEM158, FAM171A1, and SFRP1, and the third group comprises NFIB and SFRP1, and
(ii) calculating patient-specific data from the combination of levels determined from the at least two biomarkers.

Alternatively, the method of predicting the response of a breast cancer patient to a chemotherapy can be worded as comprising the step of:

calculating patient-specific data from a combination of levels determined/obtained from at least two biomarkers in a biological sample of the breast cancer patient, wherein the at least two biomarkers are selected from three groups, the at least two (e.g. 2, 3, 4, 5, 6, 7, 8, 9, 10, 11, or 12) biomarkers belonging to different groups, wherein the three groups comprise a first group,
a second group, and a third group, wherein
the first group comprises SCUBE2, CA12, and ANXA9,
the second group comprises ELF5, ROPN1, ROPN1B, SOX10, TMEM158, FAM171A1, and SFRP1, and
the third group comprises NFIB and SFRP1.

In one embodiment, the patient-specific data is calculated using a function ƒ of the combination of the levels determined from the at least two biomarkers, preferably from the at least three biomarkers, more preferably from the biomarkers SCUBE2, ELF5, and NFIB.

Preferably, the function ƒ is a function of g1, g2, and g3, wherein

g1 represents the level of the at least one biomarker of the first group,
g2 represents the level of the at least one biomarker of the second group, and
g3 represents the level of the at least one biomarker of the third group.
g1, g2, and g3, may be normalized and/or dimensionless.

More preferably, the function ƒ is a function of c1*g1+c2*g2+c3*g3 (i.e. a linear combination), wherein

c1 represents a coefficient for the level of the at least one biomarker of the first group,
c2 represents a coefficient for the level of the at least one biomarker of the second group, and
c3 represents a coefficient for the level of the at least one biomarker of the third group.

In one embodiment, the function ƒ considers reference data of a reference group. The reference group preferably comprises breast cancer patients which have been treated with chemotherapy. Said patients are patients which have clinically responded as well as patients which have clinically not responded to said therapy.

The coefficients c1, c2, and c3 may be obtained from the reference data of the reference group. Thus, the coefficient may incorporate information on clinical responders and clinical non-responders from a reference group which may be used to predict the response of a patient.

In one embodiment, the reference data is based on the same combination of levels determined from the at least two biomarkers, preferably from the at least three biomarkers, more preferably from the biomarkers SCUBE2, ELF5, and NFIB from subjects of a reference group.

Preferably, the patient-specific data is a score which is indicative for the response of the breast cancer patient to chemotherapy, particularly for the probability that the patient will respond to chemotherapy. The score allows to predict whether the patient tested will respond to chemotherapy or not. The score may be a numerical value.

Thus, in one particular embodiment, the method of predicting the response of a breast cancer patient to a chemotherapy comprises the steps of:
(i) providing a combination of levels determined from at least two biomarkers in a biological sample of the breast cancer patient,
   wherein the at least two biomarkers are selected from three groups, the at least two (e.g. 2, 3, 4, 5, 6, 7, 8, 9, 10, 11, or 12) biomarkers belonging to different groups, wherein the three groups comprise a first group, a second group, and a third group, wherein
   the first group comprises SCUBE2, CA12, and ANXA9,
   the second group comprises ELF5, ROPN1, ROPN1B, SOX10, TMEM158, FAM171A1, and SFRP1, and
   the third group comprises NFIB and SFRP1, and
(ii) calculating a score from the combination of levels determined from the at least two biomarkers.

Alternatively, the method of predicting the response of a breast cancer patient to a chemotherapy can be worded as comprising the step of:

calculating a score from a combination of levels determined/obtained from at least two biomarkers in a biological sample of the breast cancer patient, wherein the at least two biomarkers are selected from three groups, the at least two (e.g. 2, 3, 4, 5, 6, 7, 8, 9, 10, 11, or 12) biomarkers belonging to different groups, wherein the three groups comprise a first group,
a second group, and a third group, wherein
the first group comprises SCUBE2, CA12, and ANXA9,
the second group comprises ELF5, ROPN1, ROPN1B, SOX10, TMEM158, FAM171A1, and SFRP1, and
the third group comprises NFIB and SFRP1.

A test to predict the response of a breast cancer patient to chemotherapy using the biomarkers SCUBE2, ELF5, and NFIB may take place as follows:

The levels (expression levels) of the biomarkers are designated with g:

gᵢ,i∈1, . . . ,3,

where
g1: SCUBE2
g2: ELF 5
g3: NFIB
   The levels are normalized and, thus, are dimensionless, i.e. they do not have associated units. The normalization is preferably carried out using Affymetrix packages (“affy”). More preferably, the packages: affy (Version 1.44.0), Biobase (Version 2.26.0), and/or BiocGenerics (Version 0.12.1) are used.
   To map the values of the three biomarkers to the two categories “responder” and “non-responder”, a function ƒ is chosen satisfying any and/or any arbitrary combinations of the following features:

without loss of generality ƒ takes values between 0.0 and 1.0

the image of ƒ describes a sigmoid curve

as an example for a sigmoid curve, a logit function may be used

ƒ is injective

ƒ is surjective

ƒ is bijective

In case of three biomarkers g1; g2; g3, the logit function may have the form:

$$f ( g 1 , g 2 , g 3 ) = \frac{1}{1 + {e}^{- ( c 0 + c 1 g 1 + c 2 g 2 + c 3 g 3 )}} \\ c 0 = 1.76 , c 1 = 0.31 , c 2 = - 0.16 , c 3 = - 0.37 \\ \begin{matrix}c 0 : & constant intercept \\ c 1 : & coefficient of the level of SCUBE 2 \\ c 2 : & coefficient of the level of ELF 5 \\ c 3 : & coefficient of the level of NFIB\end{matrix}$$

The coefficients of the levels and c0 may be determined by mathematical/statistical evaluation of the reference data which has known clinical responders and known clinical non-responders to the chemotherapy, such that the function ƒ is fitted ideally to the reference data, e.g. by an optimization process such as by linear optimization. Therefore, the method by Broyden, Fletcher, Goldfarb and Shanno (1-bfgs) may be used. Thus, based on the reference data, a prediction may be made by calculating a score using the function ƒ. The score may be the value of the function ƒ for the patient specific levels g1, g2, g3 using the coefficients mentioned above.

Consequently, the result of the calculation is a score which allows to predict the response of the breast cancer patient to chemotherapy.

To finally make the decision whether the patient is regarded as a “responder” or “non-responder”, e.g. a patient which should be treated or a patient which should not be treated, a specific threshold parameter ξ is selected within the value range off

ξ∈[0.0,1.0], in particular ξ∈[0.2,0.7].

In the case of ξ∈[0.2, 0.7],

∀gi∈ [symbol] ⁺,i␣{1, . . . ,3}: ƒ(g1,g2,g3)≥ξ⇒“responder” (1)

β(g1,g2,g3)<ξ⇒“non-responder” (2).

Thus, a biological probe of the breast cancer patient characterized by the levels of the biomarkers SCUBE2 (g1), ELF5 (g2) and NFIB (g3) is regarded to respond to the chemotherapy and, thus, belongs to a “responder”, if

ƒ(g1,g2,g3)≥ξ.

A biological probe of the breast cancer patient characterized by the levels of the biomarkers SCUBE2 (g1), ELF5 (g2) and NFIB (g3) is regarded to not respond to the chemotherapy and, thus, belongs to a “non-responder”, if

ƒ(g1,g2,g3)<ξ.

The range from which ξ is chosen, here [0.0, 1.0] and preferably [0.2, 0.7], is expediently chosen as the range having the highest economic impact with respect to the number of patients treated and the achieved response rate/probability. Depending on which specific value of ξ is chosen, the response probability for a person regarded as “responder” may be varied depending on whether the focus is on treating as many patients as possible (lower response probability is sufficient) or whether the treatment should be as effective as possible (higher response probability is required). FIG. 6B, for example, shows sensitivity, specificity, positive predictive power (PPV), negative predictive power (NPV) for different threshold values of J.

Preferably, the chemotherapy comprises the administration of a taxane. In particular, the taxane is paclitaxel or docetaxel.

More preferably, the response is a pathological complete response (pCR).

The biological sample may be a tissue sample, e.g. tumor tissue sample (obtainable e.g. by biopsy) or a body fluid sample. The body fluid sample may be blood or a blood component (e.g. blood cells, plasma, or serum).

In one embodiment, the biological sample is a breast tumor sample. In particular, the breast tumor sample is a pre-treatment breast tumor sample. It is preferably obtained from a patient which is treatment naïve with regard to breast cancer.

In one another embodiment, the breast cancer is HER2-negative breast cancer.

In one another embodiment, the levels determined from the at least two biomarkers are levels of the RNA transcripts of said at least two biomarkers. In particular, the levels are expression levels.

In a second aspect, the present invention relates to the (in vitro) use of a combination of levels determined from at least two biomarkers in a biological sample of a breast cancer patient for predicting the response of the breast cancer patient to a chemotherapy,

wherein the at least two biomarkers are selected from three groups, the at least two biomarkers belonging to different groups, wherein the three groups comprise a first group, a second group, and a third group, wherein
the first group comprises SCUBE2, CA12, and ANXA9,
the second group comprises ELF5, ROPN1, ROPN1B, SOX10, TMEM158, FAM171A1, and SFRP1, and
the third group comprises NFIB and SFRP1.

Preferably, the at least two biomarkers belonging to different groups and differ from each other. This means that it is not possible to select SFRP1 from the second group and SFRP1 from the third group.

For example,

one biomarker is selected from the first group and another biomarker is selected from the second group,
one biomarker is selected from the first group and another biomarker is selected from the third group, or
one biomarker is selected from the second group and another biomarker is selected from the third group.

In one preferred embodiment,

the biomarker SCUBE2 is selected from the first group and the biomarker ELF5 is selected from the second group,
the biomarker SCUBE2 is selected from the first group and the biomarker NFIB is selected from the third group, or
the biomarker ELF5 is selected from the second group and the biomarker NFIB is selected from the third group.

It is also possible to select more than one biomarker from a single group under the proviso that at least two biomarkers from different groups are selected.

In one more preferred embodiment, the combination of levels is determined from at least three biomarkers, at least one first biomarker, at least one second biomarker, and at least one third biomarker, wherein

the at least one first biomarker is selected from the first group consisting of SCUBE2, CA12, and ANXA9,
the at least one second biomarker is selected from the second group consisting of ELF5, ROPN1, ROPN1B, SOX10, TMEM158, FAM171A1, and SFRP1, and the at least one third biomarker is selected from the third group consisting of NFIB and SFRP1.

In one even more preferred embodiment, the at least one first biomarker is SCUBE2, the at least one second biomarker is ELF5, and the at least one third biomarker is NFIB.

CA12 and ANXA9 are biomarkers which correlate with SCUBE2 and, thus, they may be used in addition or alternatively to SCUBE2. ROPN1, ROPN1B, SOX10, TMEM158, FAM171A1, and SFRP1 are biomarkers which correlate with ELF5 and, thus, they may be used in addition or alternatively to ELF5. SFRP1 is a biomarker which correlates with NFIB and, thus, may be used in addition or alternatively to NFIB.

Preferably, the chemotherapy comprises the administration of a taxane. In particular, the taxane is paclitaxel or docetaxel.

More preferably, the response is a pathological complete response (pCR).

The biological sample may be a tissue sample, e.g. tumor tissue sample (obtainable e.g. by biopsy) or a body fluid sample. The body fluid sample may be blood or a blood component (e.g. blood cells, plasma, or serum).

In one embodiment, the biological sample is a breast tumor sample. In particular, the breast tumor sample is a pre-treatment breast tumor sample. It is preferably obtained from a patient which is treatment naïve with regard to breast cancer.

In one another embodiment, the breast cancer is HER2-negative breast cancer.

In one another embodiment, the levels determined from the at least two biomarkers are levels of the RNA transcripts of said at least two biomarkers. In particular, the levels are expression levels.

As to further embodiments, it is referred to the first aspect of the present invention. In a third aspect, the present invention relates to a (an in vitro) method of determining whether to treat a breast cancer patient with a chemotherapy comprising the steps of:
(i) carrying out the method according to the first aspect to obtain patient specific data,
(ii) determining whether to treat the breast cancer patient with a chemotherapy based on comparing the patient-specific data with at least one reference criterion, and
(iii) if the patient-specific data meets the at least one reference criterion recommending treatment of the patient with a chemotherapy.

In one embodiment, the reference criterion is chosen considering the desired probability that the breast cancer patient responds to the chemotherapy and/or the number of breast cancer patients available to be treated. The desired probability may set to be within an interval of >0% and 100%. The desired probability is preferably set within an interval of between 5 and 100%, and more preferably of between 10 and 80%, e.g. 1, 2, 3, 4, 5, 6, 7, 8, 9, 10, 15, 20, 25, 30, 35, 40, 45, 50, 55, 60, 65, 70, 75, 80, 85, 90, 95, 99, or 100%. The reference criterion is preferably chosen as having the highest economic impact with respect to the number of patients treated and the achieved response rate. It delimits a treatment interval from a non-treatment interval.

The choice of the reference criterion generally depends on the question, whether the method of predicting the response of a breast cancer patient to a chemotherapy should have a high sensitivity or specificity or whether sensitivity and specificity should be equally weighted. FIG. 6B, for example, shows sensitivity, specificity, positive predictive power (PPV), and negative predictive power (NPV) for different reference criteria.

The sensitivity refers to the ability of the method to correctly identify patients as responder. A test with 100% sensitivity, therefore, correctly identifies all patients as responder. A test with 80% sensitivity detects 80% of the patients as responder (right-positive), but 20% of the responder remain undetected (wrong-negative). High sensitivity is particularly important for screening purposes.
The specificity refers to the ability of the method to correctly identify patients as non-responders. A test with 100% specificity, therefore, correctly identifies all non-responders. A test with 80% specificity identifies 80% of non-responders as test negative (true negative), but 20% of the non-responders are falsely identified as test positive (false positive). For each test there is usually a compromise between the two values. This compromise can be represented graphically with the aid of a receiver operating characteristic (ROC) curve (see, for example, FIG. 6B). With the method of the present invention, high sensitivity as well as high specificity values could be reached (see examples for further information).

In one embodiment, the reference criterion is a reference cut-off/threshold.

In one embodiment, the patient-specific data is a score. When the score is within the treatment interval, the breast cancer patient has a probability to respond which is greater than a predefined probability, or when the score is within the non-treatment interval, the patient has a probability to respond which is less than the predefined probability. The predefined probability is preferably set within the interval of between 5 and 100%, and more preferably of between 10 and 80%, e.g. 1, 2, 3, 4, 5, 6, 7, 8, 9, 10, 15, 20, 25, 30, 35, 40, 45, 50, 55, 60, 65, 70, 75, 80, 85, 90, 95, 99, or 100%. ξ may be indicative for the predefined probability. When the score is within the treatment interval, treatment of the patient with chemotherapy is recommended, or when the score is within the non-treatment interval, treatment of the patient with chemotherapy is not recommended. It should be noted that the predefined probability is the desired probability.

The range of the reference cut-off/threshold (e.g. designated as is preferably chosen as having the highest economic impact with respect to the number of patients treated and the achieved response rate. It delimits a treatment interval from a non-treatment interval.

It is preferred that the reference cut-off/threshold is a value within the value range off preferably between 0.0 and 1, more preferably between 0.2 and 0.7, even more preferably between 0.21 and 0.68.

FIG. 6B, for example, shows sensitivity, specificity, positive predictive power (PPV), negative predictive power (NPV) for different threshold values of ξ. The meaning of the cut-off/threshold (e.g. designated as ξ) is obvious when selecting the boundary values, e.g. 0.0 and 1.0. If the cut-off/threshold is set to 0.0, this suggests that all patients are considered as potential “responders” while no patient is excluded as a potential “non-responder”. In this case, there is no difference of carrying out Companion diagnostics (CDx) and not carrying out Companion diagnostics (CDx). In other words, the conduction of the method of the present invention has no advantage over the usual procedure of no Companion diagnostic (CDx) assay. At this threshold, the sensitivity, which is defined by the fraction of true responders by all responders, is exactly sensitivity=1.0. Increasing the cut-off/threshold (e.g. designated as j) increases the PPV while at the same time the sensitivity decreases as potential responders are lost which are wrongly classified as “non-responders”. If the cut-off/threshold is set to 1.0, the model classifies all patients as non-responders which yields the highest specificity. The specificity is the fraction of true predicted non-responders by the number of all non-responders.
Obviously, the external points of the range of 0.0 and 1.0 are not useful. In a preferred embodiment, the cut-off/threshold is set to be between 0.2 and 0.7 as this range has the highest economic impact with respect to the number of patients treated and the achieved response rate.

In the case of ξ∈[0.2, 0.7], preferably of ξ∈[0.21, 0.68],

∀gi∈ [symbol] ⁺,i∈{1, . . . ,3}: ƒ(g1,g2,g3)≥ξ⇒“responder” (1)

ƒ(g1,g2,g3)<ξ⇒“non-responder” (2).

Thus, a biological probe of the breast cancer patient characterized by the levels of the biomarkers SCUBE2 (g1), ELF5 (g2) and NFIB (g3) is regarded to respond to the chemotherapy and, thus, belongs to a “responder”, if

ƒ(g1,g2,g3)≥ξ.

A biological probe of the breast cancer patient characterized by the levels of the biomarkers SCUBE2 (g1), ELF5 (g2) and NFIB (g3) is regarded to not respond to the chemotherapy and, thus, is a “non-responder”, if

ƒ(g1,g2,g3)<ξ.

The range from which ξ is chosen, here [0.0, 1.0], preferably [0.2, 0.7], and more preferably [0.21, 0.68], is expediently chosen as the range having the highest economic impact with respect to the number of patients treated and the achieved response rate/probability. Depending on which specific value of ξ is chosen the response probability for a person regarded as “responder” may be varied depending on whether the focus is on treating as many patients as possible (lower response probability is sufficient) or whether the treatment should be as effective as possible (higher response probability is required). FIG. 6B, for example, shows sensitivity, specificity, positive predictive power (PPV), negative predictive power (NPV) for different threshold values of ξ.

Preferably, the chemotherapy comprises the administration of a taxane. More preferably, the taxane is paclitaxel or docetaxel.

As to further embodiments of the method of the third aspect of the present invention, it is referred to the first aspect of the present invention.

In a fourth aspect, the present invention relates to a (an in vitro) method of predicting the response of a breast cancer patient to a chemotherapy comprising the step of: determining the level of at least one biomarker selected from the group consisting of SCUBE2 and ELF5 in a biological sample of a breast cancer patient.

The level of the biomarker NFIB may be further determined in the biological sample of the breast cancer patient.

Thus, in one preferred embodiment,

the level of the biomarker SCUBE2 and the level of the biomarker NFIB,
the level of the biomarker SCUBE2 and the level of the biomarker ELF5, or
the level of the biomarker ELF5 and the level of the biomarker NFIB
is determined in the biological sample of the breast cancer patient.

In one more preferred embodiment, the level of the biomarker SCUBE2, the level of the biomarker ELF5, and the level of the biomarker NFIB is determined in the biological sample of the breast cancer patient.

In one even more preferred embodiment, the level of the at least one biomarker is compared to a reference level of said at least one biomarker. Thus, in one particular embodiment, the method of predicting the response of a breast cancer patient to a chemotherapy comprises the steps of:
(i) determining the level of at least one biomarker selected from the group consisting of SCUBE2 and ELF5 in a biological sample of a breast cancer patient, and
(ii) comparing the level of the at least one biomarker to a reference level of said at least one biomarker.
   The above comparison allows to predict, whether the patient will respond to chemotherapy or not.

The reference level may be any level which allows to determine whether a patient will respond to chemotherapy or not. It may be obtained from (a) (control) subject(s) (i.e. (a) subject(s) different from the individual to be tested such as (a) subject(s) known to not responded to chemotherapy (non-responder(s)) or known to responded to chemotherapy (responder(s)).

It is preferred that the reference level is the level determined by measuring at least one reference biological sample, e.g. at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 250, 300, 400, 500, or 1.000 reference biological sample(s), from at least one subject known to not responded to chemotherapy, e.g. from at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 250, 300, 400, 500, or 1.000 subject(s) known to not responded to chemotherapy. It is more preferred that the reference level is the level determined by measuring between 2 and 500 reference biological samples from between 2 and 500 subjects known to not responded to chemotherapy. It is even more preferred that the reference level is determined by measuring between 50 and 500 reference biological samples from between 50 and 500 subjects known to not responded to chemotherapy. It is most preferred that the reference level is determined by measuring between 100 and 500 reference biological samples from between 100 and 500 subjects known to not responded to chemotherapy.

In one most preferred embodiment,
(i) the level of SCUBE2 which is below the reference level indicates that the patient will respond to chemotherapy, or
   the level of SCUBE2 which is comparable with reference level indicates that the patient will not respond to chemotherapy,
(ii) the level of ELF5 which is above the reference level indicates that the patient will respond to chemotherapy, or
   the level of ELF5 which is comparable with the reference level indicates that the patient will not respond to chemotherapy, and/or
(iii) the level of NFIB which is above the reference level indicates that the patient will respond to chemotherapy, or
   the level of NFIB which is comparable with the reference level indicates that the patient will not respond to chemotherapy.

In one most preferred embodiment (alternative),
(i) the patient is regarded to respond to the chemotherapy and, thus, belongs to a responder, when the level of SCUBE2 is below the reference level, or
   the patient is regarded to not respond to the chemotherapy and, thus, belongs to a non-responder, when the level of SCUBE2 is comparable with the reference level,
(ii) the patient is regarded to respond to the chemotherapy and, thus, belongs to a responder, when the level of ELF5 is above the reference level, or
   the patient is regarded to not respond to the chemotherapy and, thus, belongs to a non-responder, when the level of ELF5 is comparable with the reference level, and/or
(iii) the patient is regarded to respond to the chemotherapy and, thus, belongs to a responder, when the level of NFIB is above the reference level, or
   the patient is regarded to not respond to the chemotherapy and, thus, belongs to a non-responder, when the level of NFIB is comparable with the reference level.

It is alternatively preferred that the reference level is the level determined by measuring at least one reference biological sample, e.g. at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 250, 300, 400, 500, or 1.000 reference biological sample(s), from at least one subject known to responded to chemotherapy, e.g. from at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 250, 300, 400, 500, or 1.000 subject(s) known to responded to chemotherapy. It is more preferred that the reference level is the level determined by measuring between 2 and 500 reference biological samples from between 2 and 500 subjects known to responded to chemotherapy. It is even more preferred that the reference level is determined by measuring between 50 and 500 reference biological samples from between 50 and 500 subjects known to responded to chemotherapy. It is most preferred that the reference level is determined by measuring between 100 and 500 reference biological samples from between 100 and 500 subjects known to responded to chemotherapy.

In one most preferred embodiment,
(i) the level of SCUBE2 which is above the reference level indicates that the patient will not respond to chemotherapy, or
   the level of SCUBE2 which is comparable with reference level indicates that the patient will respond to chemotherapy,
(ii) the level of ELF5 which is below the reference level indicates that the patient will not respond to chemotherapy, or
   the level of ELF5 which is comparable with the reference level indicates that the patient will respond to chemotherapy, and/or
(iii) the level of NFIB which is below the reference level indicates that the patient will not respond to chemotherapy, or
   the level of NFIB which is comparable with the reference level indicates that the patient will respond to chemotherapy.

In one most preferred embodiment (alternative),
(i) the patient is regarded to respond to the chemotherapy and, thus, belongs to a responder, when the level of SCUBE2 is comparable with the reference level, or
   the patient is regarded to not respond to the chemotherapy and, thus, belongs to a non-responder, when the level of SCUBE2 is above the reference level,
(ii) the patient is regarded to respond to the chemotherapy and, thus, belongs to a responder, when the level of ELF5 is comparable with the reference level, or
   the patient is regarded to not respond to the chemotherapy and, thus, belongs to a non-responder, when the level of ELF5 is below the reference level, and/or
(iii) the patient is regarded to respond to the chemotherapy and, thus, belongs to a responder, when the level of NFIB is comparable with the reference level, or
   the patient is regarded to not respond to the chemotherapy and, thus, belongs to a non-responder, when the level of NFIB is below the reference level.

A level which is “comparable with” the reference level in this respect means that the level is no more than 15%, preferably no more than 10%, more preferably no more than 5%, above the reference level or the level is no more than 15%, preferably no more than 10%, more preferably no more than 5%, below the reference level.

Alternatively, a level which is “comparable with” the reference level in this respect means that the detected level variation is within the accuracy of a measurement. The accuracy of a measurement depends on the measurement method used.

Preferably, the level of the at least one biomarker is at least 0.6-fold or 0.7-fold, more preferably at least 0.8-fold or 0.9-fold, even more preferably at least 1.2-fold or 1.5-fold, and most preferably at least 2.0-fold or 3.0-fold below/above the reference level. For example, the level of the at least one biomarker is at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

It is practicable to take one reference biological sample per subject for analysis. If additional reference biological samples are required, e.g. to determine the reference level in different reference biological samples, the same subject may be (re)tested. Said reference level may be an average reference level. It may be determined by measuring reference levels and calculating the “average” value (e.g. mean, median or modal value) thereof. It is preferred that the reference biological sample is from the same source (e.g. tissue sample) than the biological sample isolated from the patient. It is further preferred that the reference level is obtained from a subject of the same gender (e.g. female) and/or of a similar age/phase of life (e.g. adults or elderly) than the patient to be tested.

More preferably, the chemotherapy comprises the administration of a taxane. In particular, the taxane is paclitaxel or docetaxel.

Even more preferably, the response is a pathological complete response (pCR).

The level determined from the at least one biomarker is preferably a level of the RNA transcript of said at least one biomarker. Methods to determine the level of the RNA transcript in a biological sample are well known. The level of the RNA transcript is usually measured by polymerase chain reaction (PCR), in particular by reverse transcription quantitative polymerase chain reaction (RT-PCR and qPCR) or real-time PCR. RT-PCR is used to create a cDNA from the mRNA. The cDNA may be used in a qPCR assay to produce fluorescence as the DNA amplification process progresses. This fluorescence is proportional to the original mRNA amount in the samples. Other methods to be used include Microarray, Northern blots, Fluorescence in situ hybridization (FISH), microarrays, and RT-PCR combined with capillary electrophoresis. The level is preferably an expression level.

The biological sample used to determine the level of the at least one biomarker may be a tissue sample, e.g. tumor tissue sample (obtainable e.g. by biopsy) or a body fluid sample. The body fluid sample may be blood or a blood component (e.g. blood cells, plasma, or serum).

In one embodiment, the biological sample is a breast tumor sample. In particular, the breast tumor sample is a pre-treatment breast tumor sample. It is preferably obtained from a patient which is treatment naïve with regard to breast cancer.

In one another embodiment, the breast cancer is HER2-negative breast cancer.

In a fifth aspect, the present invention relates to (the use of) a kit for predicting the response of a breast cancer patient to a chemotherapy comprising means for determining the level of at least one biomarker selected from the group consisting of SCUBE2 and ELF5 in a biological sample of a breast cancer patient.

Preferably, the kit is used in vitro.

The kit may further comprise means for determining the level of the biomarker NFIB in the biological sample of the breast cancer patient.

Thus, in one preferred embodiment, the kit comprises means for determining

the level of the biomarker SCUBE2 and the level of the biomarker NFIB,
the level of the biomarker SCUBE2 and the level of the biomarker ELF5, or
the level of the biomarker ELF5 and the level of the biomarker NFIB
in the biological sample of the breast cancer patient.

In one more preferred embodiment, the kit comprises means for determining

the level of the biomarker SCUBE2,
the level of the biomarker ELF 5, and
the level of the biomarker NFIB
in the biological sample of the breast cancer patient.

Said means may be probes or primer pairs allowing the detection of the above mentioned biomarkers, preferably on RNA transcript, in particular mRNA, level.

Preferably, the kit comprises at least one biomarker-specific (in particular RNA transcript) primer pair and/or at least one biomarker-specific (in particular RNA transcript) probe.

More preferably, the kit comprises

at least one SCUBE2-specific primer pair and/or at least one SCUBE2-specific probe and at least one NFIB-specific primer pair and/or at least one NFIB-specific probe,
at least one SCUBE2-specific primer pair and/or at least one SCUBE2-specific probe and at least one ELF5-specific primer pair and/or at least one ELF5-specific probe,
at least one ELF5-specific primer pair and/or at least one ELF5-specific probe and at least one NFIB-specific primer pair and/or at least one NFIB-specific probe, or
at least one SCUBE2-specific primer pair and/or at least one SCUBE2-specific probe, at least one NFIB-specific primer pair and/or at least one NFIB-specific probe, and at least one ELF5-specific primer pair and/or at least one ELF5-specific probe.

Even more preferably, the kit comprises at least one reference which allows to predict whether the patient will respond or not respond to chemotherapy. The at least one reference may be a reference level. For each biomarker tested, a respective reference level may be required. The reference may also be a cut-off/threshold which allows to predict whether the patient will respond or not respond to chemotherapy. For each biomarker tested, a respective cut-off/threshold may be required.

In one embodiment, the kit is useful for conducting the method according to the fourth aspect of the present invention.

In one embodiment, the kit further comprises

(i) a container, and/or
(ii) a data carrier.

Said data carrier may be a non-electronical data carrier, e.g. a graphical data carrier such as an information leaflet, an information sheet, a bar code or an access code, or an electronical data carrier such as a floppy disk, a compact disk (CD), a digital versatile disk (DVD), a microchip or another semiconductor-based electronical data carrier. The access code may allow the access to a database, e.g. an internet database, a centralized, or a decentralized database. The access code may also allow access to an application software that causes a computer to perform tasks for computer users or a mobile app which is a software designed to run on smartphones and other mobile devices.

Said data carrier may further comprise at least one reference which allows to predict whether the patient will respond or not respond to chemotherapy. The at least one reference may be a reference level. For each biomarker tested, a respective reference level may be required. The reference may also be a cut-off/threshold which allows to predict whether the patient will respond or not respond to chemotherapy. For each biomarker tested, a respective cut-off/threshold may be required.
In case that the data carrier comprises an access code which allows the access to a database, said at least one reference is deposited in this database.

The data carrier may comprise instructions on how to carry out the method according to the fourth aspect.

Said kit may also comprise materials desirable from a commercial and user standpoint including a buffer(s), a reagent(s) and/or a diluent(s) for determining the level mentioned above.

Preferably, the chemotherapy comprises the administration of a taxane. In particular, the taxane is paclitaxel or docetaxel.

More preferably, the response is a pathological complete response (pCR).

The biological sample may be a tissue sample, e.g. tumor tissue sample (obtainable e.g. by biopsy) or a body fluid sample. The body fluid sample may be blood or a blood component (e.g. blood cells, plasma, or serum). In one embodiment, the biological sample is a breast tumor sample. In particular, the breast tumor sample is a pre-treatment breast tumor sample. It is preferably obtained from a patient which is treatment naïve with regard to breast cancer.

In one another embodiment, the breast cancer is HER2-negative breast cancer.

In one another embodiment, the levels determined from the at least two biomarkers are levels of the RNA transcripts of said at least two biomarkers. In particular, the levels are expression levels.

In a further aspect, the present invention relates to a method of predicting the response of a breast cancer patient to a chemotherapy based on a combination of levels determined from the biomarkers

### (i) ILF2, CXCR4, and WWP1,

### (ii) IGHG1, IGHG3, IGHM, IGHV4-31, ID4, and CSRP2, or

(iii) DNAJC12, PRSS23, and TTC39A
in a biological sample of the breast cancer patient.

In a further aspect, the present invention relates to the use of a combination of levels determined from the biomarkers

### (i) ILF2, CXCR4, and WWP1,

### (ii) IGHG1, IGHG3, IGHM, IGHV4-31, ID4, and CSRP2, or

(iii) DNAJC12, PRSS23, and TTC39A
in a biological sample of a breast cancer patient
for predicting the response of the breast cancer patient to a chemotherapy.

In the above further aspects, it is preferred that the chemotherapy comprises the administration of a taxane such as paclitaxel or docetaxel.

It is further preferred that the levels determined from the biomarkers are expression levels of the RNA transcripts of said biomarkers.
It is also preferred that the response is a pathological complete response (pCR).
It is more preferred that the biological sample is a breast tumor sample such as a pre-treatment breast tumor sample.
It is even more preferred that the breast cancer is HER2-negative breast cancer.

As to further preferred embodiments, it is referred to the first and second aspect described herein.

The present invention is further summarized as follows:
1. A method of predicting the response of a breast cancer patient to a chemotherapy based on a combination of levels determined from at least two biomarkers in a biological sample of the breast cancer patient,
   wherein the at least two biomarkers are selected from three groups, the at least two biomarkers belonging to different groups, wherein the three groups comprise a first group, a second group, and a third group, wherein
   the first group comprises SCUBE2, CA12, and ANXA9,
   the second group comprises ELF5, ROPN1, ROPN1B, SOX10, TMEM158, FAM171A1, and SFRP1, and
   the third group comprises NFIB and SFRP1.
2. The method of item 1, wherein the combination of levels is determined from at least three biomarkers, at least one first biomarker, at least one second biomarker, and at least one third biomarker, wherein
   the at least one first biomarker is selected from the first group consisting of SCUBE2, CA12, and ANXA9,
   the at least one second biomarker is selected from the second group consisting of ELF5, ROPN1, ROPN1B, SOX10, TMEM158, FAM171A1, and SFRP1, and
   the at least one third biomarker is selected from the third group consisting of NFIB and SFRP1.
3. The method of item 2, wherein the at least one first biomarker is SCUBE2, the at least one second biomarker is ELF5, and the at least one third biomarker is NFIB.
4. The method of any one of items 1 to 3, wherein the combination of levels determined from the at least two biomarkers comprises a linear combination of levels, wherein the levels in the linear combination are weighted differently.
5. The method of any one of items 1 to 4, wherein the method comprises the step of calculating patient-specific data from the (linear) combination of levels determined from the at least two biomarkers.
6. The method of item 5, wherein the patient-specific data is calculated using a function ƒ of the combination of the levels determined from the at least two biomarkers.
7. The method of item 6, wherein the function ƒ is a function of g1, g2, and g3, wherein
   g1 represents the level of the at least one biomarker of the first group,
   g2 represents the level of the at least one biomarker of the second group, and
   g3 represents the level of the at least one biomarker of the third group.
8. The method of item 7, wherein the function ƒ is a function of c1*g1+c2*g2+c3*g3, wherein
   c1 represents a coefficient for the level of the at least one biomarker of the first group,
   c2 represents a coefficient for the level of the at least one biomarker of the second group, and
   c3 represents a coefficient for the level of the at least one biomarker of the third group.
9. The method of any one of items 6 to 8, wherein the function ƒ considers reference data of a reference group.
10. The method of item 9, wherein the reference data is based on the same combination of levels determined from the at least two biomarkers from subjects of a reference group.
11. The method of any one of items 5 to 10, wherein the patient-specific data is a score which is indicative for the response of the breast cancer patient to chemotherapy.
12. The method of any one of items 1 to 11, wherein the chemotherapy comprises the administration of a taxane.
13. The method of item 12, wherein the taxane is paclitaxel or docetaxel.
14. The method of any one of items 1 to 13, wherein the levels determined from the at least two biomarkers are levels of the RNA transcripts of said at least two biomarkers.
15. The method of any one of items 1 to 14, wherein the levels are expression levels.
16. The method of any one of items 1 to 15, wherein the response is a pathological complete response (pCR).
17. The method of any one of items 1 to 16, wherein the biological sample is a breast tumor sample.
18. The method of item 17, wherein the breast tumor sample is a pre-treatment breast tumor sample.
19. The method of any one of items 1 to 18, wherein the breast cancer is HER2-negative breast cancer.
20. Use of a combination of levels determined from at least two biomarkers in a biological sample of a breast cancer patient for predicting the response of the breast cancer patient to a chemotherapy,
   wherein the at least two biomarkers are selected from three groups, the at least two biomarkers belonging to different groups, wherein the three groups comprise a first group, a second group, and a third group, wherein
   the first group comprises SCUBE2, CA12, and ANXA9,
   the second group comprises ELF5, ROPN1, ROPN1B, SOX10, TMEM158, FAM171A1, and SFRP1, and
   the third group comprises NFIB and SFRP1.
21. The use of item 20, wherein the combination of levels is determined from at least three biomarkers, at least one first biomarker, at least one second biomarker, and at least one third biomarker, wherein
   the at least one first biomarker is selected from the first group consisting of SCUBE2, CA12, and ANXA9,
   the at least one second biomarker is selected from the second group consisting of ELF5, ROPN1, ROPN1B, SOX10, TMEM158, FAM171A1, and SFRP1, and
   the at least one third biomarker is selected from the third group consisting of NFIB and SFRP1.
22. The use of item 21, wherein the at least one first biomarker is SCUBE2, the at least one second biomarker is ELF5, and the at least one third biomarker is NFIB.
23. The use of any one of items 20 to 22, wherein the chemotherapy comprises the administration of a taxane.
24. The use of item 23, wherein the taxane is paclitaxel or docetaxel.
25. The use of any one of items 20 to 24, wherein the levels determined from the at least two biomarkers are levels of the RNA transcripts of said at least two biomarkers.
26. The use of any one of items 20 to 25, wherein the levels are expression levels.
27. The use of any one of items 20 to 26, wherein the response is a pathological complete response (pCR).
28. The use of any one of items 20 to 27, wherein the biological sample is a breast tumor sample.
29. The use of item 28, wherein the breast tumor sample is a pre-treatment breast tumor sample.
30. The use of any one of items 20 to 29, wherein the breast cancer is HER2-negative breast cancer.
31. A method of determining whether to treat a breast cancer patient with a chemotherapy comprising the steps of:
   (i) carrying out the method of any one of items 1 to 20 to obtain patient specific data,
   (ii) determining whether to treat the breast cancer patient with a chemotherapy based on comparing the patient-specific data with at least one reference criterion, and
   (iii) if the patient-specific data meets the at least one reference criterion recommending treatment of the patient with a chemotherapy.
32. The method of item 31, wherein the reference criterion is chosen considering the desired probability that the breast cancer patient responds to the chemotherapy and/or the number of breast cancer patients available to be treated.
33. The method of items 31 or 32, wherein the reference criterion is a reference threshold which delimits a treatment interval from a non-treatment interval.
34. The method of item 33, wherein the reference threshold is a value within the value range of ƒ.
35. The method of any one of items 31 or 34, wherein the patient-specific data is a score and wherein,
   when the score is within the treatment interval, the breast cancer patient has a probability to respond which is greater than a predefined probability, or
   when the score is within the non-treatment interval, the patient has a probability to respond which is less than the predefined probability.
36. The method of item 35, wherein
   when the score is within the treatment interval, treatment of the patient with chemotherapy is recommended, or
   when the score is within the non-treatment interval, treatment of the patient with chemotherapy is not recommended.
37. The method of items 35 or 36, wherein the predefined probability is the desired probability.
38. A method of predicting the response of a breast cancer patient to a chemotherapy comprising the step of:
   determining the level of at least one biomarker selected from the group consisting of SCUBE2 and ELF5 in a biological sample of a breast cancer patient.
39. The method of item 38, wherein level of the biomarker NFIB is further determined in the biological sample of the breast cancer patient.
40. The method of item 39, wherein the level of the biomarker SUBE2, the level of the biomarker ELF5, and the level of the biomarker NFIB is determined in the biological sample of the breast cancer patient.
41. The method of any one of items 38 to 40, wherein the level of the at least one biomarker is compared to a reference level of said at least one biomarker.
42. The method of item 41, wherein the reference level is the level determined by measuring at least one reference biological sample from at least one subject known to not responded to chemotherapy.
43. The method of items 41 or 42, wherein
   (i) the level of SCUBE2 which is below the reference level indicates that the patient will respond to chemotherapy, or
      the level of SCUBE2 which is comparable with the reference level indicates that the patient will not respond to chemotherapy,
   (ii) the level of ELF5 which is above the reference level indicates that the patient will respond to chemotherapy, or
      the level of ELF5 which is comparable with the reference level indicates that the patient will not respond to chemotherapy, and/or
   (iii) the level of NFIB which is above the reference level indicates that the patient will respond to chemotherapy, or
      the level of NFIB which is comparable with the reference level indicates that the patient will not respond to chemotherapy.
44. The method of any one of items 38 to 43, wherein the chemotherapy comprises the administration of a taxane.
45. The method of item 44, wherein the taxane is paclitaxel or docetaxel.
46. The method of any one of items 38 to 45, wherein the level determined from the at one biomarker is a level of the RNA transcript of said at least one biomarker.
47. The method of any one of items 38 to 46, wherein the level is an expression level.
48. The method of any one of items 38 to 47, wherein the response is a pathological complete response (pCR).
49. The method of any one of items 38 to 48, wherein the biological sample is a breast tumor sample.
50. The method of item 49, wherein the breast tumor sample is a pre-treatment breast tumor sample.
51. The method of any one of items 38 to 50, wherein the breast cancer is HER2-negative breast cancer.
52. A kit for predicting the response of a breast cancer patient to a chemotherapy comprising means for determining the level of at least one biomarker selected from the group consisting of SCUBE2 and ELF5 in a biological sample of a breast cancer patient.
53. The kit of item 52, wherein the kit further comprises means for determining the level of the biomarker NFIB in the biological sample of the breast cancer patient.
54. The kit of item 53, wherein the kit comprises means for determining
   the level of the biomarker SUBE2,
   the level of the biomarker ELF5, and
   the level of the biomarker NFIB
   in the biological sample of the breast cancer patient.
55. The kit of items 52 to 54, wherein the kit is useful for conducting the method according to any one of items 38 to 51.
56. The kit of any one of items 52 to 55, wherein the kit further comprises
   (i) a container, and/or
   (ii) a data carrier.
57. The kit of item 56, wherein the data carrier comprises instructions on how to carry out the method according to any one of items 38 to 51.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

The following Figures and Examples are merely illustrative of the present invention and should not be construed to limit the scope of the invention as indicated by the appended claims in any way.

### BRIEF DESCRIPTION OF THE FIGURES

FIG. 1: Applied biomarker workflow.

FIG. 2: A PCA of the already normalized data (RD group) of the discovery cohort showing a site effect between the data obtained at the I-SPY-1 and MDACC sites, respectively (upper panel (A)). The same figure applying quantile normalization to the discovery set (lower panel, (B)).

FIG. 3: Normalized data of discovery and validation cohorts according to the normalization prescription of the present inventors, see text for details (upper panel (A)). Signal distribution of the discovery cohort (lower panel (B)). Relative frequencies are shown such that the histogram shapes may be compared more easily. pCR=predict pathologic complete response, RD=non-responder.

FIG. 4: Bar graphs of the genes contained in the gene signature of the classification model comparing the responder class (pCR=predict pathologic complete response) with the non-responder class (RD). The case of the validation cohort is shown.

FIG. 5: Histograms of the genes contained in the gene signature of the classification model comparing the distributions within the responder class with the distributions within the non-responder class within the validation cohort.

FIG. 6: The receiver-operator characteristics curve of our model comparing the performances on the discovery and validation sets (upper panel (A)). Various performance characteristics depending on the chosen model-internal threshold used for predicting responders (lower panel (B)).

FIG. 7: The receiver-operator characteristics curves of our model comparing the site specific performances. The LBJ (panel (A)) and USO (panel (B)) site curves demonstrate the cross site validation performance of our classification model, while the MDACC site result (1 panel) is shown for completeness (panel (C)).

FIG. 8: Comparison of response rate of the model of the present inventors (OakLabs) to the cases without Companion diagnostic (CDx) assay and with the model by Hatzis et al.

### EXAMPLES

### 1. Methods

CDx Workflow

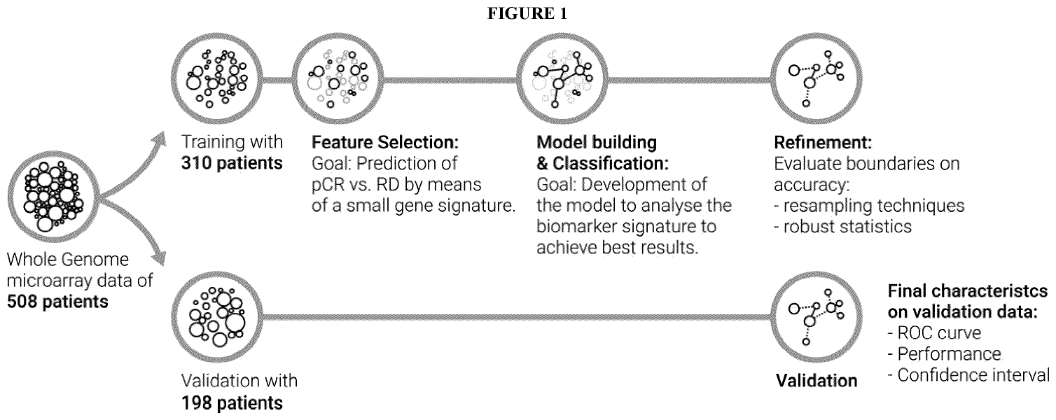

In FIG. 1 summarizes the steps that are indispensable for a successful predictive biomarker study with maximum probability for obtaining a market-ready, approved companion diagnostic. Starting at taking (genome-wide) molecular data—already in the early stages of the study—and after proper data preprocessing (whitening) signals in the registered data can be readily analyzed and a robust gene signature for discerning non-responders from responders to the drug under study is developed. Further, a model is built on the basis of the underlying gene signature data and maximized for best performance. The optimal model is then refined and validated on an independent set of samples.

Choice of Data Set for Case Study

As prerequisite for a reliable and robust machine learning analysis the underlying data sets should fulfil the following points: a high enough total number of samples (a); from independent study sites (b); with global gene expression profiles publicly available (e.g. by ArrayExpress) (c); a single technology platform used for data acquisition (d); data taken at baseline of the study (e); sufficient meta data to access and work with (f); published in a high ranking journal (g). The first point is crucial in order to be able to split up the data into a discovery and a validation set, which is inevitable for a reliable machine learning analysis, since any algorithm needs to be validated with data that has not been used in the training phase in order to allow to assess its performance and in order to circumvent the well-known overfitting problem. Points (b) and (c) allow free access to the complete gene profile of patients while point (d) ensures that data is intrinsically comparable. Point (e) is necessary to ensure that differences in the responders and non-responders are not caused by the applied medical treatment. Point (f): it is important to have the means to understand possible patterns found while analyzing the data that are not caused by the treatment—think of lab effects, effects due to patient sex, age dependence etc.

The data obtained in the study undertaken by Hatzis et al. [11] of HER2 negative breast cancer patients meets all of the above listed criteria. The pretreatment characteristics of the discovery cohort of 310 patients and the validation cohort of 198 patients have been reported. The data has been taken prior to taxane-anthracycline based chemotherapy. All data has been acquired using the U133A GeneChip by Affymetrix. No details are available for the sequential taxane and anthracycline based chemotherapy for individual patients as well as on the question which patients of the validation cohort received entirely neoadjuvant (N=165), partial neoadjuvant (N=18) or entirely adjuvant (N=16) chemotherapy.

Evaluation of Data Quality and Data Processing

Gene expression raw data were obtained from the ArrayExpress online pages (IDs: E-GEOD-25055[13] and E-GEOD-25065[14]). They provide both the already normalized sets of the data as have been provided by the authors of [11] as well as the respective raw data.

It is widely accepted that gene expression based data requires a proper normalization of the recorded data for comparing samples among each other. On the other hand, it should be assured that the normalization method does not introduce clusters when normalizing data from different sites, different cohorts etc. This is even more true if the data is used to train a classification algorithm since it might learn a pattern that has been artificially introduced by the normalization.

### 2. Results

Unbiased Sample Normalization

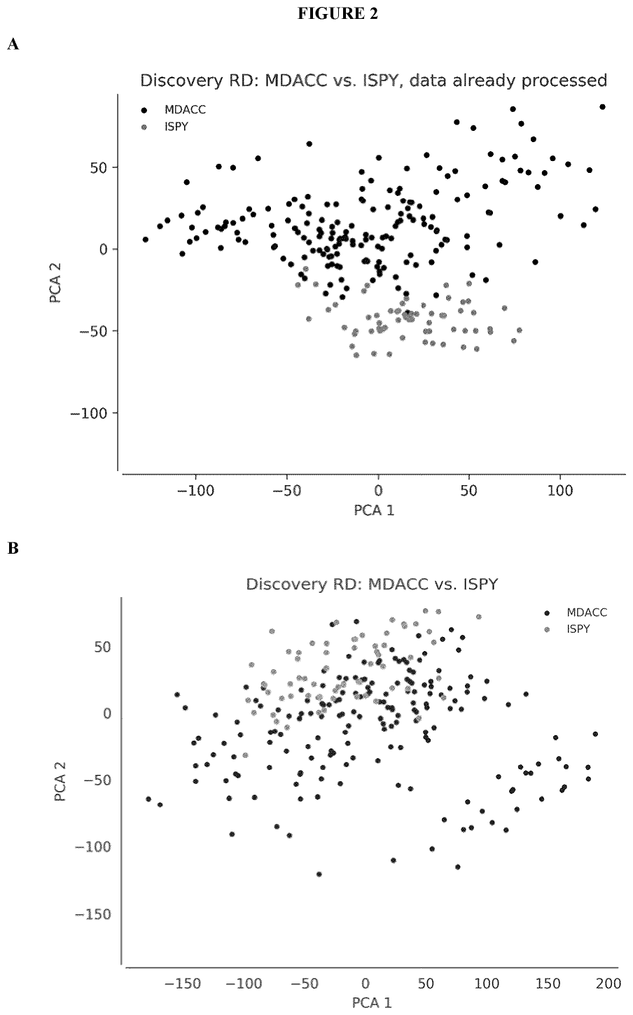

As a possible starting point for model selection and for training the machine learning algorithms, the already normalized data provided via ArrayExpress were considered. However, it turned out that this data contains patterns revealed using a primary component analysis (PCA) when comparing the two sites I-SPY-1 and MDACC data, cf. the upper panel (A) of FIG. 2, where the RD group of the data is shown, as more samples are available in this group. The data projected on the two major axes in the PCA space is shown.

In an attempt to possibly eliminate or at least reduce the splitting between the two labs, the data were normalized using the Affymetrix package (“affy”) available for the R programming language to normalize the discovery cohort. Many options are available and provided by the “affy” package to process the input gene chip data using R. The “rma” method for background correction, quantile normalization using the “quantile” option, “pmonly” for only using the signals of the pm channel as well as “medianpolish” for data summary were employed. Although the effect does not completely vanish even when using this alternative normalization, the site-specific clustering in the PCA of the discovery cohort was significantly reduced as can be seen from the lower panel (B) of FIG. 2.

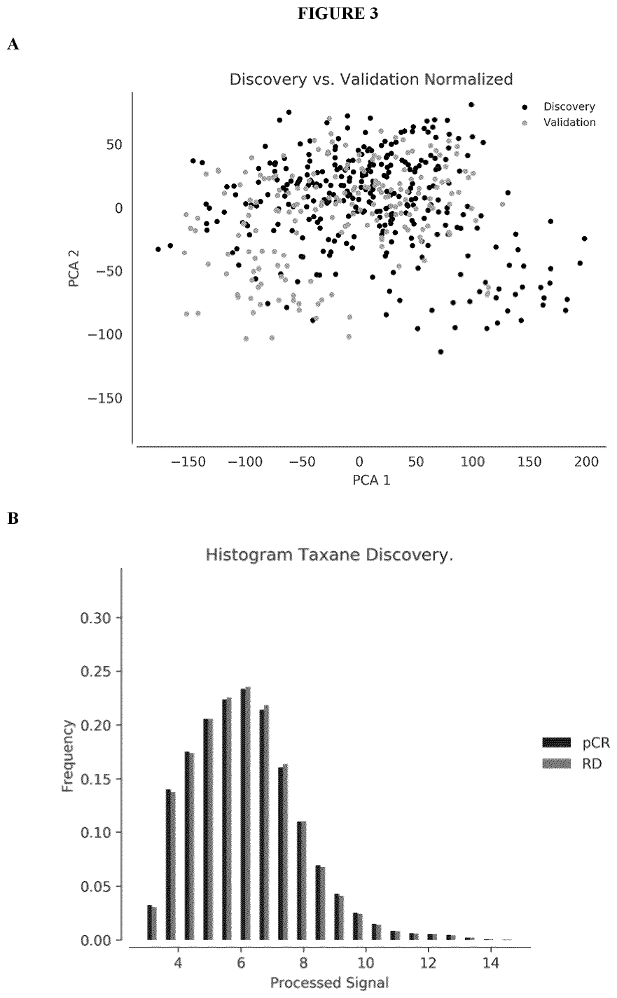

A known disadvantage of the quantile normalization is that it does not treat a single sample on its own, but the signals of all samples together are used to adapt the signal of each individual sample. In a machine learning context, this behavior of the normalization is disfavorable since a clear separation of the data used for training and tuning the model from the data used to validate the model is always mandatory in order to prevent overfitting. The validation of a model should be done using data that was unknown during the learning phase. In order to have normalized validation cohort samples which moreover are maximally independent of one another, it was chosen to normalize each validation cohort sample together with all available discovery cohort samples. In this way, it was possible to retain the advantages of quantile normalization also for the validation cohort, but avoid spilling validation cohort information into the discovery cohort, thus, guaranteeing independence of the training phase from the validation data. As an additional benefit, this method makes it possible to easily validate new samples with the already trained model and setup. The upper panel (A) of FIG. 3 shows that using this way of normalizing the validation cohort samples, no clustering is visible comparing the normalized discovery data to the normalized validation data in a PCA analysis. Thus, the two cohorts of data after the chosen normalization are compatible and are ready to be used as input to subsequent machine learning analysis.

Finally, it was also checked for the discovery cohort that after normalization a similar distribution of signals for the pCR and RD groups is present, cf. the lower panel (B) of FIG. 3 (pCR=pathological complete response, RD=residual invasive disease/non-responder).

Feature Selection and Classification Model

A major part in the development of a machine learning algorithm is the choice of an appropriate feature set (gene signature). A notorious problem in life sciences as compared to other fields, where AI algorithms are commonly applied, is the limited amount of samples (N) and the high costs related to each sample. On the other hand, the number of recorded features (genes, proteins etc.) M is usually much higher than N. Since after all, a machine learning algorithm is based in one way or another on a fitting i.e. regression technique, it is crucial to reduce the gene set sufficiently in order to have some degrees of freedom left within the fit. The feature set can be reduced using filters (for instance filtering on p-value or the mean signal), removing redundant i.e. highly correlated genes, using classifier based methods (such as RFE [15]) or using L1-norm based lasso techniques [16]. In addition, biological input concerning the mode of action of the drug and associated pathways is valuable to reduce the set further. A brute-force search over all possible gene subsets may also be done if starting from a sufficiently small feature set and using parallel computing techniques in order to overcome the associated 2^{M} asymptotics.

It was made use of all above mentioned techniques to single out the optimal gene signature that allows the training of a performant classification algorithm and is yet small in size. It is however of utter importance to use proper resampling methods to obtain in average performance comparisons of different gene signatures. Resampling as well as cross-validation techniques on the discovery cohort were used to obtain robust metrics.

One may choose among a plethora of classification algorithms that are available on the market, such as linear models, tree-based models (with or without boosting), different kinds of support vector machines, network based classifiers etc. Each of them has its own right of existence and comes with its strengths and weaknesses. For example, some are good at capturing non-linear effects, others perform worse in such cases. Furthermore, there are algorithms that tend to more easily overfit than others. It is therefore crucial to understand the underlying data (noise, variance, reproducibility between labs) to choose the best classification model. Several classifiers were tested on our candidate gene signatures in order to choose the best performing algorithm. Thereby, the mean value of the achieved performance metric as well as strive for a low variation i.e. a low standard deviation, were taken into account. A summary of the tested classification models and their achieved performances in shown in Table 1.

Table 1 Examples of classifier performances in order to illustrate the need to test several algorithms. The achieved mean ROC area under the curve score and its standard error (68% CI) are shown.

| Candidate Algorithm | AUC score |
|---|---|
| Decision Tree | 0.77 (0.06) |
| Logistic Regression | 0.82 (0.05) |
| Radial basis function SVM | 0.82 (0.04) |
| LogitBoost | 0.80 (0.06) |

The area under the curve (AUC) of the receiver-operator characteristics (ROC) was chosen as the most appropriate score method for the unbalanced data sets underlying this study. The commonly used accuracy would require a balancing of the unbalanced data within the two classes. Here, the comparison of the classifiers using a cross-validation technique on the whole discovery set was performed fixing the same ratio of training and test number of samples at 0.7 in each case. The achieved mean and standard error of the AUC were reported.

Performance of the Predictive Biomarker Signature in the Independent Validation Cohort

The final model, whose performance is the subject of this paragraph, is based on merely the three genes listed in Table 2.

TABLE 2
Gene signature

| Affymetrix Code | Gene Symbol |
|---|---|
| X219197_s_at | SCUBE2 |
| X220625_s_at | ELF5 |
| X209289_at | NFIB |

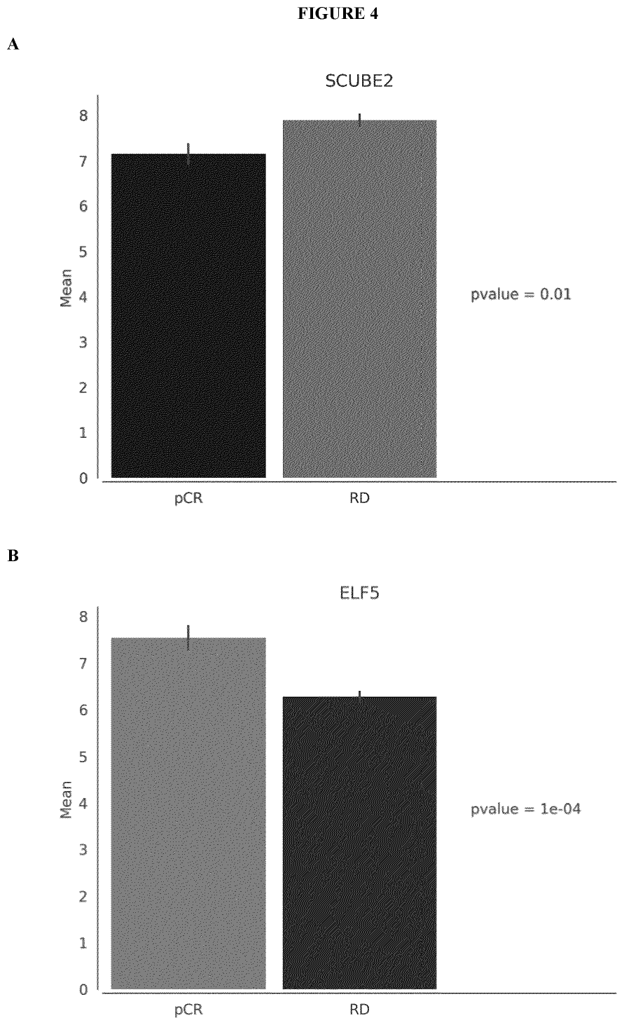

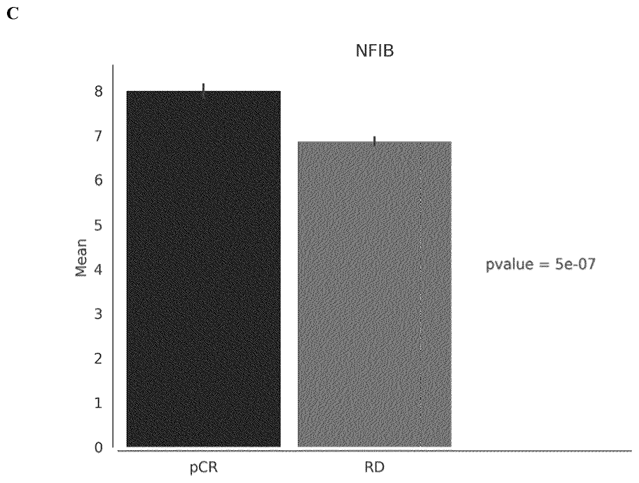

FIG. 4 shows bar graphs of the genes contained in the gene signature (SCUBE 2, ELF5, and NFIB) of the classification model comparing the responder class (pCR=predict pathologic complete response) with the non-responder class (RD). The case of the validation cohort is shown.

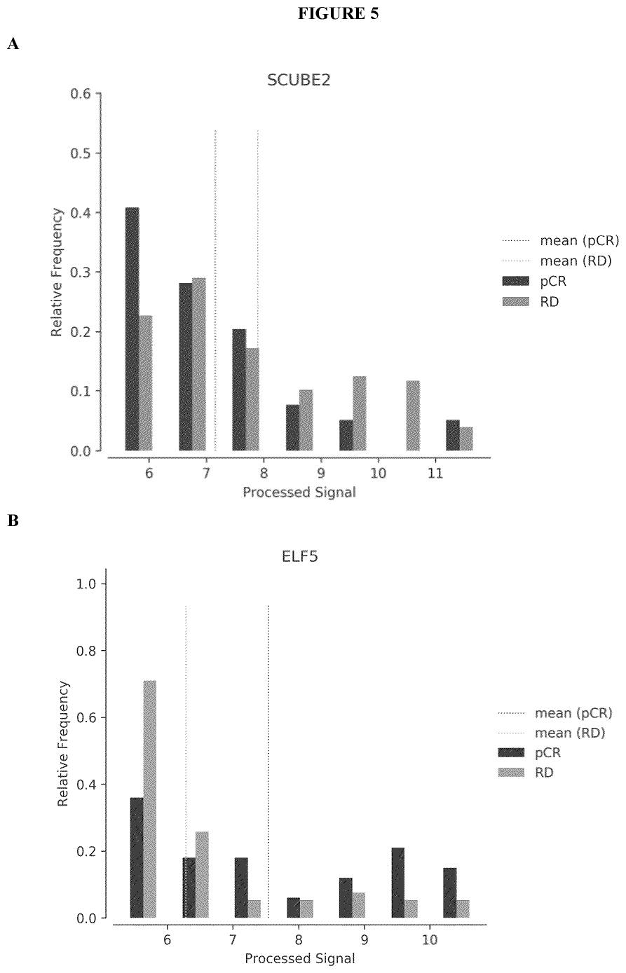

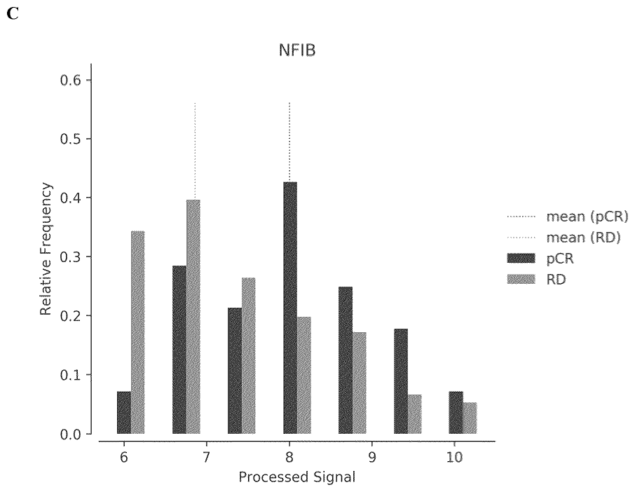

Comparing the histograms of these genes within the responder's group with the histograms within the non-responders group shown in FIG. 5 reveals that there are indeed differences even on the level of a single gene.

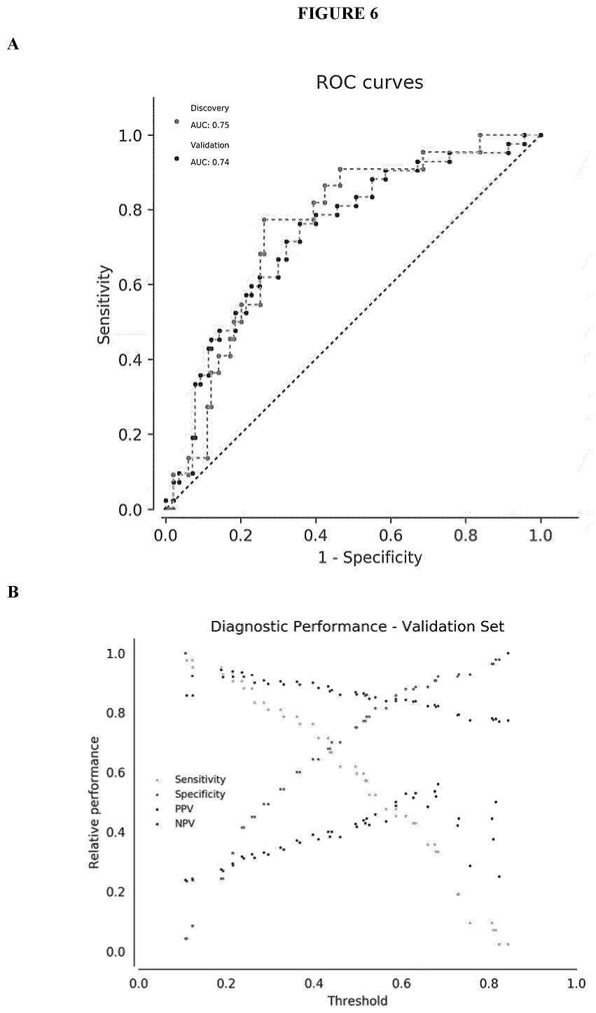

The model's performance on the discovery and validation cohorts is presented in the upper panel (A) of FIG. 6, where the ROC curve is shown. The curve as obtained on the discovery set as well as the curve obtained when predicting the unknown validation cohort data is shown. Various performance characteristics depending on the chosen model-internal threshold used for predicting responders are shown in the lower panel (B) of FIG. 6. It can be observed from the figure, both ROC are compatible, from which it is concluded that a valid classification model was developed, which would not be the case if either of the curve would deviate significantly from the other.

The performance of the new model for predicting pCR and RD was next compared with the model by Hatzis et al. [11]. The available meta data published with the data has been used to obtain them. In Table 3 the various model performance metrics were accumulated.

Table 3 Comparison of response prediction algorithm performance on the independent validation cohort (182 samples). The sensitivity of our model has been matched to the value of Hatzis et al. by setting the ROC work point to 0.520. See text for further details. CDx=Companion diagnostics, positive predictive value (PPV), negative predictive value (NPV).

| | Without CDx | Hatzis et al. | Present analysis |
|---|---|---|---|
| Response rate | 23% | 33% | 44% |
| Sensitivity | — | 55% | 57% |
| Specificity | — | 67% | 79% |
| PPV | — | 33% | 44% |
| NPV | — | 83% | 86% |

The response rate without Companion diagnostics (CDx) (defined in the usual way as fraction of responders and all patients treated) and with CDx, which is equal to the positive predictive value (PPV) were also computed. Since all performance values are mutually dependent and furthermore depend intrinsically on the operation point of the classifier, i.e. the model-internal threshold for the probability of classifying “responders”, one can only compare two models if either of the performance metrics matches among the models. Our classifier's operation point was set to a value of 0.520 such that our achieved sensitivity matches as closely as possible the sensitivity of the model by Hatzis et al. Doing so, the model is completely fixed and the specificity, the positive predictive value (PPV), and the negative predictive value (NPV) in Table 3 are directly comparable. For reference, the dependence of the performance numbers in the lower panel (B) of FIG. 6 were additionally visualized.

Similarly, the operation point of our model may be chosen such that instead the specificity is matched to the one obtained by Hatzis et al., which is approximately obtained at a value of 0.437. The other performance characteristics in this case may be read from Table 4 and may be compared among the two models.

Table 4 Comparison of response prediction algorithm performance on the independent validation cohort (182 samples). The specificity of our model has been matched to the value of Hatzis et al. by setting the ROC work point to 0.437. See text for further details. CDx=Companion diagnostics, positive predictive value (PPV), negative predictive value (NPV).

| | Without CDx | Hatzis et al. | Present analysis |
|---|---|---|---|
| Response rate | 23% | 33% | 38% |
| Sensitivity | — | 55% | 68% |
| Specificity | — | 67% | 68% |
| PPV | — | 33% | 38% |
| NPV | — | 83% | 87% |

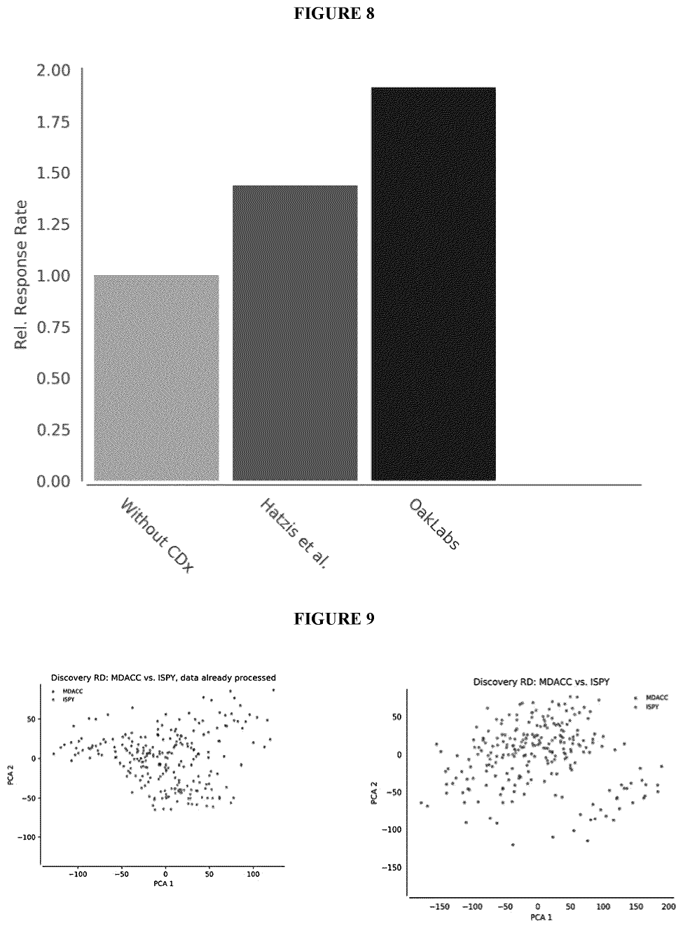

The response rates of the cases without companion diagnostics, the Hatzis et al. model and the model of the present inventors evaluated at a threshold 0.520 is visualized in FIG. 8.

Cross-Site Validation

In order to rule out medical site-related biases of our model, a cross-site validation study was performed. Since data taken at the MDACC site has been included both in the discovery cohort and the validation cohort, while samples from the LBJ/INEN/GEICAM (for brevity called LBJ in what follows) and USO centers are only included in the validation cohort of the original study, the easiest way to accomplish a cross-site validation may be to predict only the data of the latter two sites.

In this way data originating from the same medical centers in both the learning and application stages of our model could be avoided, while still being able to compare our model's performance on the validation set with values reported in literature, as has been done above. The site-specific performances that are achieved by our model as presented above are summarized in Table 5.

Table 5 Cross-site study performance of our model at the ROC work point 0.520. The 95% confidence intervals are shown in parenthesis. See text for details. CDx=Companion diagnostics, positive predictive value (PPV), negative predictive value (NPV).

| | | | | | ROC |
|---|---|---|---|---|---|
| Site | Sensitivity | Specificity | PPV | NPV | AUC |
| LBJ/INEN/ | 45(27)% | 85(11)% | 42(22)% | 87(7)% | 0.73 |
| GEICAM | | | | | |
| USO | 64(22)% | 66(16)% | 51(15)% | 77(11)% | 0.65 |
| MDACC | 58(25)% | 82(10)% | 40(18)% | 91(5)% | 0.82 |
| all sites | 57(14)% | 78(7)% | 44(11)% | 86(4)% | 0.74 |

For reference, the performances obtained for all sites together were also included. Additionally, the 95% confidence interval of our model performance numbers was computed using a bootstrapping scheme over the validation cohort samples. Symmetrical 95% confidence intervals were found. The associated errors are listed in Table 3 in parenthesis. As can be seen, the values obtained for the USO and LBJ sites are compatible with the average obtained including all sites.

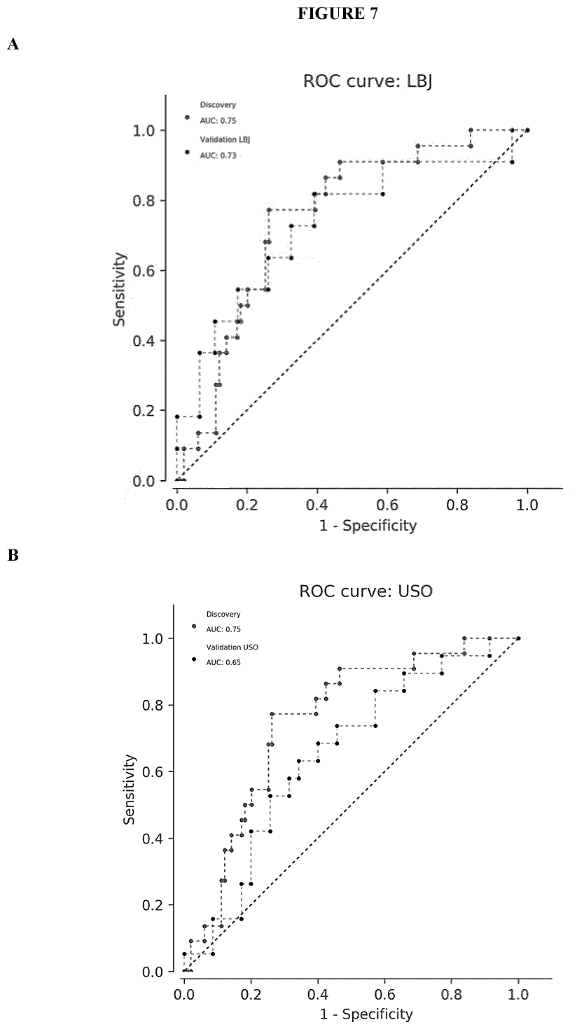

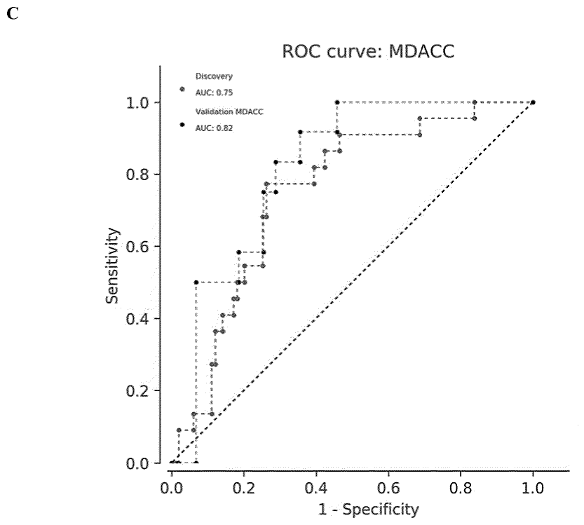

The site-specific ROC curves shown in FIG. 7 were achieved. Comparing the AUC scores, is has been observed that for the LBJ site (panel (A)) a similarly good score was obtained, while for the USO site (panel (B)) a slightly worse value was obtained, which has dropped by 0.1 as compared to the discovery set. Given the typical fluctuations, an AUC of about 0.04-0.06 at the 68% confidence level was observed (cf. Table 1). The drop is insignificant. The performance on the MDACC validation set on the other hand, which was added for completeness, is slightly better than the reference discovery value (panel (C)).

Example of a Mathematical Calculation with the Novel Signature of the Biomarkers SCUBE2, ELF5, and NFIB in Order to Predict Whether a Patient Will Respond to Chemotherapy or not

In the following, a mathematical calculation with the novel signature of the biomarkers SCUBE2, ELF5, and NFIB is shown in order to predict whether a patient will respond to chemotherapy or not:

The levels (expression levels) of the biomarkers SCUBE2, ELF5, and NFIB were designated with g:

gi,i∈E1, . . . ,3,

where
g1: SCUBE2
g2: ELF 5
g3: NFIB
   The levels were normalized and are, thus, dimensionless, i.e. they do not have associated units. The normalization was carried out using the Affymetrix packages (“affy”). More specifically, the packages: affy (Version 1.44.0), Biobase (Version 2.26.0), BiocGenerics (Version 0.12.1) were used.
   To map the values of the three biomarkers to the two categories “responder” and “non-responder”, a function ƒ was chosen satisfying any and/or any arbitrary combinations of the following features:

without loss of generality ƒ takes values between 0.0 and 1.0

the image of ƒ describes a sigmoid curve

as an example for a sigmoid curve, a logit function may be used

ƒ is injective

ƒ is surjective

ƒ is bijective

In case of three biomarkers g1; g2; g3, a logit function was selected having the form:

$$f ( g 1 , g 2 , g 3 ) = \frac{1}{1 + {e}^{- ( c 0 + c 1 g 1 + c 2 g 2 + c 3 g 3 )}} \\ c 0 = 1.76 , c 1 = 0.31 , c 2 = - 0.16 , c 3 = - 0.37 \\ \begin{matrix}c 0 : & constant intercept \\ c 1 : & coefficient of the level of SCUBE 2 \\ c 2 : & coefficient of the level of ELF 5 \\ c 3 : & coefficient of the level of NFIB\end{matrix}$$

The coefficients of the levels and c0 were determined by mathematical/statistical evaluation of the reference data which has known clinical responders and known clinical non-responders to the chemotherapy, such that the function ƒ is fitted ideally to the reference data, e.g. by an optimization process such as by linear optimization. The method by Broyden, Fletcher, Goldfarb and Shanno (1-bfgs) was used here. Thus, based on the reference data, a prediction is made by calculating a score using the function ƒ. The score is the value of the function ƒ for the patient specific levels g1, g2, g3 using the coefficients mentioned above.
Consequently, the result of the calculation is a score which allows to predict the response of the breast cancer patient to chemotherapy.
To finally make the decision whether the patient is regarded as a “responder” or “non-responder”, e.g. a patient which should be treated or a patient which should not be treated, a specific threshold parameter ξ is selected within the value range off:

ξ∈[0.0,1.0].

In case of E [0.0, 1.0],

∀gi∈ [symbol] ⁺,i∈{1, . . . ,3}: ƒ(g1,g2,g3)≥ξ⇒responder (1)

ƒ(g1,g2,g3)<ξ⇒non-responder (2).

FIG. 6 shows the sensitivity, specificity, positive predictive value (PPV) and the negative predictive value (NPV) for different values of the threshold parameter ξ.
In fact, the new response rate of Taxane restricted to the patients which are predicted responders by this model is identical to the PPV. The sensitivity denotes the fraction of true responders and all responders.
The meaning of the parameter ξ is obvious when selecting the external values. If ξ is set to ξ=0.0 equation, this suggests that all patients are considered as potential responders while no patient is excluded as a potential non-responder. In this case the PPV should match the actual response rate (23%) of the Taxane without any Companion diagnostics (CDx) which is given at PPV=0.23. At this threshold, the sensitivity, which is defined by the fraction of true responders by all responders, is exactly sensitivity=1.0. Increasing the parameter ξ increases the PPV while at the same time the sensitivity decreases as responders are lost which are wrongly classified as non-responders. At ξ=1.0, the model classifies all patients as non-responders which yields the highest specificity. The specificity is the fraction of true predicted non-responders by the number of all non-responders.
Obviously, the extremal points of ξ are not useful. The plot shows that ξ should be chosen between 0.2 and 0.7 as this range has the highest economic impact with respect to the number of patients treated and the achieved response rate.

0.2≤ƒ(g1,g2,g3)≤0.7,gi∈ [symbol] ⁺,i∈1, . . . ,3

Here specific statistics for a selection of ξ at the limits of the above range ξ∈[0.2, 0.7] assuming a total number of 1.000 patients are given:

### Without Companion Diagnostics (CDx):

Total patients: 1.000
Response rate: 0.23 (=23%)

### Responders: 230

### Non-Responders: 770

### With ξ=0.21:

Total patients: 1.000
Response rate (PPV): 0.29 (=29%)
Predicted responders: 728

### True Responders: 214

### Non-Responders: 515

### With ξ=0.68:

Total patients: 1.000
Response rate (PPV): 0.56 (=56%)
Predicted responders: 137

### Responders: 77

### Non-Responders: 60

Further, the following ratios could be reached:
SCUBE2: mean (R)/mean (NR)=0.86+/−0.06
ELF5: mean (R)/mean (NR)=1.22+/−0.02
NFIB: mean (R)/mean (NR)=1.18+/−0.02

### R=Responder

### NR=Non-Responder

SCUBE2, ELF5, and NFIB and their Correlated Genes

The signature comprising the genes SCUBE2, ELF5, and NFIB was determined. In addition, correlated genes of SCUBE2, ELF5, and NFIB were identified. The level of said genes can alternatively be measured/determined.
SCUBE2 (geneID=57758′) and its correlated genes CA12 (gene_id=771) and ANXA9 (gene_id=8416),
ELF5 (geneID=2001′) and its correlated genes ROPN1 (gene_id=54763), ROPN1B (gene_id=152015), SOX10 (gene_id=6663), TMEM158 (gene_id=25907), FAM171A1 (gene_id=221061), and SFRP1 (gene_id=6422), and
NFIB (geneID=4781′) and its correlated gene SFRP1 (gene_id=6422).
SCUBE2, ELF5, and NFIB are genes.
Two genes are said to be correlated if their variation about their respective mean values is not statistically independent, but mutually and linearly related. The Pearson correlation coefficient, which normalizes the expectation value of the common variation about the mean value of the genes with the product of the standard deviations of the two gene's signals, has been used here.
In addition to the signature(s) described above, the following signatures were determined/calculated:

### 1. ILF2, CXCR4, and WWP1,

### 2. IGHG1, IGHG3, IGHM, IGHV4-31, ID4, and CSRP2, or

### 3. DNAJC12, PRSS23, and TTC39A.

They allow the prediction of the response of a breast cancer patient to chemotherapy. The prediction response (e.g. with respect to sensitivity and/or specificity) was, however, not as good as for the signature(s) described above.

### 3. Discussion

In this study, results in life sciences were improved by using dedicated new AI concepts. A well suited case example of high medical relevance in the field of breast cancer was chosen and demonstrated the superiority of our approach: With a model of just 3 genes the response rate can almost be increased by 33% compared to the benchmark published by Hatzis et al.

Having evolved in the field of image recognition, artificial intelligence and machine learning algorithms are increasingly employed for tasks in life sciences. While images are highly reproducible and contain several million data points (pixels), life science data are quite different in respect to number of data points and noise for example. Algorithms in image recognition require approximations to deliver results within minutes. In contrast, the major demand on predictive biomarkers is maximum ac-curacy. This can only be achieved by complete avoidance of approximations which in turn increases the computing time. Two months of computing time on a compute cluster with 80 compute cores were necessary for our results.

The majority of public genome-wide gene expression data is not compatible with an approach to develop reliable predictive biomarkers, mainly due to limitations in sample size. An integrative analysis of raw data from independent studies could improve the situation, but comes with a number of challenges. Differences in the experimental protocols or technology platform used can introduce systematic variation across studies. The focus here was on gene expression data of sufficient samples obtained on a single technology platform with minimal variation in the experimental protocols. Such a setting could easily be implemented as part of a clinical phase 3 and is compatible with a straightforward translation of the developed biomarker signature to a companion diagnostics assay.

An interdisciplinary team of quantum physicists and life scientists was able to develop and cross-site validate a 3-genes predictive biomarker signature which is capable of nearly doubling the response rate within the group of predicted responders.

Adding strength to our results is that all three genes are biologically plausible. They all are described in the literature in the context of cancer and breast cancer in particular. SCUBE2 (Signal peptide-complement protein C1r/C1s, Uegf, and Bmp1 [CUB]-epidermal growth factor [EGF] domain-containing protein) is an 807-amino acids protein that belongs to a small family of three members. SCUBE2 is predominantly expressed in vascular endothelial cells [17] and regulates the SHH (Sonic Hedgehog) signaling, acting upstream of ligand binding at the plasma membrane [18]. Mounting evidence suggests that SCUBE2 acts as a tumor suppressor in breast cancer [19,20], NSCLC [21], colorectal cancer [22] and gastric cancer [23].

ELF5 (E74 Like E26 transformation-specific [ETS] Transcription Factor 5) is a 265-amino acids protein and a member of the ETS family of transcription factors. ETS family proteins regulate a wide spectrum of biological processes and several ETS factors have been implicated with cancer initiation, progression and metastasis [25,26]. For ELF5, both tumor promoting and suppressive roles have been reported in breast cancer [27].

NFIB belongs to the nuclear factor 1 (NFI) family of transcription factors which control expression of a large number of cellular genes [29,30]. In a hetero and homodimer complex, the four members of the NFI family can activate or repress transcription depending on the context [30]. NFIB has been defined as an oncogene in several reports [31,32]. The chromosomal region encoding NFIB is amplified in TNBC [33].

### 4. Conclusion

A novel AI-based approach enabled the development of a predictive biomarker signature that significantly outperforms the benchmark in respect to accuracy, number of features and reproducibility. The small size of the signature allows efficient translation to a CDx assay that is compatible with technology in routine diagnostic laboratories. Especially in view of increasing costs and time for clinical trials, predictive single drug biomarkers combined with modern trial designs offer the opportunity to increase the R&D productivity in healthcare.

### REFERENCES

1. Learn, P. A., Yeh, I.-T., McNutt, M., Chisholm, G. B., Pollock, B. H., Rousseau Jr, D. L., Sharkey, F. E., Cruz, A. B., Kahlenberg, M. S.: Her-2/neu expression as a predictor of response to neoadjuvant docetaxel in patients with operable breast carcinoma. Cancer: Interdisciplinary International Journal of the American Cancer Society 103(11), 2252-2260 (2005)
2. Vogel, C., Cobleigh, M., Tripathy, D., Gutheil, J., Harris, L., Fehrenbacher, L., Slamon, D., Murphy, M., Novotny, W., Burchmore, M., et al.: First-line, single-agent herceptin® (trastuzumab) in metastatic breast cancer: a preliminary report. European journal of cancer 37, 25-29 (2001)
3. Audeh, M. W., Carmichael, J., Penson, R. T., Friedlander, M., Powell, B., Bell-McGuinn, K. M., Scott, C., Weitzel, J. N., Oaknin, A., Loman, N., et al.: Oral poly (adp-ribose) polymerase inhibitor olaparib in patients with brca1 or brca2 mutations and recurrent ovarian cancer: a proof-of-concept trial. The Lancet 376(9737), 245-251 (2010)
4. Kaufman, B., Shapira-Frommer, R., Schmutzler, R. K., Audeh, M. W., Friedlander, M., Balmaña, J., Mitchell, G., Fried, G., Stemmer, S. M., Hubert, A., et al.: Olaparib monotherapy in patients with advanced cancer and a germline brca1/2 mutation. Journal of clinical oncology: official journal of the American Society of Clinical Oncology 33(3), 244 (2015)
5. Herbst, R. S., Soria, J.-C., Kowanetz, M., Fine, G. D., Hamid, O., Gordon, M. S., Sosman, J. A., McDermott, D. F., Powderly, J. D., Gettinger, S. N., et al.: Predictive correlates of response to the anti-pd-11 antibody mpd13280a in cancer patients. Nature 515(7528), 563 (2014)
6. Garon, E. B., Rizvi, N. A., Hui, R., Leighl, N., Balmanoukian, A. S., Eder, J. P., Patnaik, A., Aggarwal, C., Gubens, M., Horn, L., et al.: Pembrolizumab for the treatment of non-small-cell lung cancer. New England Journal of Medicine 372(21), 2018-2028 (2015)
7. Herbst, R. S., Baas, P., Kim, D.-W., Felip, E., Pérez-Gracia, J. L., Han, J.-Y., Molina, J., Kim, J.-H., Arvis, C. D., Ahn, M.-J., et al.: Pembrolizumab versus docetaxel for previously treated, pd-11-positive, advanced non-small-cell lung cancer (keynote-010): a randomized controlled trial. The Lancet 387(10027), 1540-1550 (2016)
8. Kim, S., Lin, C.-W., Tseng, G. C.: Metaktsp: a meta-analytic top scoring pair method for robust cross-study validation of omics prediction analysis. Bioinformatics 32(13), 1966-1973 (2016)
9. Rohart, F., Eslami, A., Matigian, N., Bougeard, S., Le Cao, K.-A.: Mint: a multivariate integrative method to identify reproducible molecular signatures across independent experiments and platforms. BMC bioinformatics 18(1), 128 (2017)
10. Harris, L. N., Ismaila, N., McShane, L. M., Andre, F., Collyar, D. E., Gonzalez-Angulo, A. M., Hammond, E. H., Kuderer, N. M., Liu, M. C., Mennel, R. G., et al.: Use of biomarkers to guide decisions on adjuvant systemic therapy for women with early-stage invasive breast cancer: American society of clinical oncology clinical practice guideline. Journal of Clinical Oncology 34(10), 1134 (2016)
11. Hatzis, C., Pusztai, L., Valero, V., Booser, D. J., Esserman, L., Lluch, A., Vidaurre, T., Holmes, F., Souchon, E., Wang, H., et al.: A genomic predictor of response and survival following taxane-anthracycline chemotherapy for invasive breast cancer. Jama 305(18), 1873-1881 (2011)
12. Bianco, S., Burger, F., Kallarackal, J., Romualdi, A., Schad, M.: Prediction of sensitivity to taxane-antracycline chemotherapy in invasive breast cancer (in preparation). TBA (2019)
13. Hatzis, C.: Discovery cohort for genomic predictor of response and survival following neoadjuvant taxane-anthracycline chemotherapy in breast cancer. https://www.ebi.ac.uk/arrayexpress/experiments/E-GEOD-25055/?query=GSE25055. [Online; accessed 5 Jun. 2019]” (2011)
14. Hatzis, C.: Validation cohort for genomic predictor of response and survival following neoadjuvant taxane-anthracycline chemotherapy in breast cancer. https://www.ebi.ac.uk/arrayexpress/experiments/E-GEOD-25065/?query=GSE25065. [Online; accessed 5 Jun. 2019] (2011)
15. Guyon, I., Weston, J., Barnhill, S., Vapnik, V.: Gene selection for cancer classification using support vector machines. Machine Learning 46(1), 389-422 (2002). doi:10.1023/A:1012487302797
16. Tibshirani, R.: Regression shrinkage and selection via the lasso: a retrospective. (2011)
17. Yang, R.-B., Ng, C. K. D., Wasserman, S. M., Colman, S. D., Shenoy, S., Mehraban, F., Kömüves, L. G., Tomlinson, J. E., Topper, J. N.: Identification of a novel family of cell-surface proteins expressed in human vascular endothelium. Journal of Biological Chemistry 277(48), 46364-46373 (2002)
18. Tsai, M.-T., Cheng, C.-J., Lin, Y.-C., Chen, C.-C., Wu, A.-R., Wu, M.-T., Hsu, C.-C., Yang, R.-B.: Isolation and characterization of a secreted, cell-surface glycoprotein scube2 from humans. Biochemical Journal 422(1), 119-128 (2009)
19. Cheng, C.-J., Lin, Y.-C., Tsai, M.-T., Chen, C.-S., Hsieh, M.-C., Chen, C.-L., Yang, R.-B.: Scube2 suppresses breast tumor cell proliferation and confers a favorable prognosis in invasive breast cancer. Cancer Research 69(8), 3634-3641 (2009)
20. Lin, Y.-C., Chen, C.-C., Cheng, C.-J., Yang, R.-B.: Domain and functional analysis of a novel breast tumor suppressor protein, scube2. Journal of Biological Chemistry 286(30), 27039-27047 (2011)
21. Yang, B., Miao, S., Li, Y.: Scube2 inhibits the proliferation, migration and invasion of human non-small cell lung cancer cells through regulation of the sonic hedgehog signaling pathway. Gene 672, 143-149 (2018)
22. Song, Q., Li, C., Feng, X., Yu, A., Tang, H., Peng, Z., Wang, X.: Decreased expression of scube2 is associated with progression and prognosis in colorectal cancer. Oncology reports 33(4), 1956-1964 (2015)
23. Wang, X., Zhong, R.-Y., Xiang, X.-J.: Reduced expression of scube2 predicts poor prognosis in gastric cancer patients. INTERNATIONAL JOURNAL OF CLINICAL AND EXPERIMENTAL PATHOLOGY 11(2), 972-980 (2018)
24. Van′t Veer, L. J., Dai, H., Van De Vijver, M. J., He, Y. D., Hart, A. A., Mao, M., Peterse, H. L., Van Der Kooy, K., Marton, M. J., Witteveen, A. T., et al.: Gene expression profiling predicts clinical outcome of breast cancer. nature 415(6871), 530 (2002)
25. Sharrocks, A. D.: The ets-domain transcription factor family. Nature reviews Molecular cell biology 2(11), 827 (2001)
26. Hsu, T., Trojanowska, M., Watson, D. K.: Ets proteins in biological control and cancer. Journal of cellular biochemistry 91(5), 896-903 (2004)
27. Luk, I., Reehorst, C., Mariadason, J.: Elf3, elf5, ehf and spdef transcription factors in tissue homeostasis and cancer. Molecules 23(9), 2191 (2018)
28. Omata, F., McNamara, K. M., Suzuki, K., Abe, E., Hirakawa, H., Ishida, T., Ohuchi, N., Sasano, H.: Effect of the normal mammary differentiation regulator elf5 upon clinical outcomes of triple negative breast cancers patients. Breast Cancer 25(4), 489-496 (2018)
29. Gronostaj ski, R. M.: Roles of the nfi/ctf gene family in transcription and development. Gene 249(1-2), 31-45 (2000)
30. Harris, L., Genovesi, L. A., Gronostaj ski, R. M., Wainwright, B. J., Piper, M.: Nuclear factor one transcription factors: divergent functions in developmental versus adult stem cell populations. Developmental dynamics 244(3), 227-238 (2015)
31. Dooley, A. L., Winslow, M. M., Chiang, D. Y., Banerji, S., Stransky, N., Dayton, T. L., Snyder, E. L., Senna, S., Whittaker, C. A., Bronson, R. T., et al.: Nuclear factor i/b is an oncogene in small cell lung cancer. Genes & development 25(14), 1470-1475 (2011)
32. Zhang, Q., Cao, L.-Y., Cheng, S.-J., Zhang, A.-M., Jin, X.-S., Li, Y.: p53-induced microrna-1246 inhibits the cell growth of human hepatocellular carcinoma cells by targeting nfib. Oncology reports 33(3), 1335-1341 (2015)
33. Han, W., Jung, E.-M., Cho, J., Lee, J. W., Hwang, K.-T., Yang, S.-J., Kang, J. J., Bae, J.-Y., Jeon, Y. K., Park, I.-A., et al.: Dna copy number alterations and expression of relevant genes in triple-negative breast cancer. Genes, Chromosomes and Cancer 47(6), 490-499 (2008)

1.-15. (canceled)

16. A method of predicting the response of a breast cancer patient to a chemotherapy
based on a combination of levels determined from at least two biomarkers in a biological sample of the breast cancer patient,
wherein the at least two biomarkers are selected from three groups, the at least two biomarkers belonging to different groups and differ from each other, wherein the three groups comprise a first group, a second group, and a third group, wherein
the first group comprises SCUBE2, CA12, and ANXA9,
the second group comprises ELF5, ROPN1, ROPN1B, SOX10, TMEM158, FAM171A1, and SFRP1, and
the third group comprises NFIB and SFRP1.

17. The method of claim 16, wherein the combination of levels is determined from at least three biomarkers, at least one first biomarker, at least one second biomarker, and at least one third biomarker, wherein
the at least one first biomarker is selected from the first group consisting of SCUBE2, CA12, and ANXA9,
the at least one second biomarker is selected from the second group consisting of ELF5, ROPN1, ROPN1B, SOX10, TMEM158, FAM171A1, and SFRP1, and
the at least one third biomarker is selected from the third group consisting of NFIB and SFRP1.

18. The method of claim 17, wherein the at least one first biomarker is SCUBE2, the at least one second biomarker is ELF5, and the at least one third biomarker is NFIB.

19. The method of claim 16, wherein the chemotherapy comprises the administration of a taxane.

20. The method of claim 19, wherein the taxane is paclitaxel or docetaxel.

21. The method of claim 16, wherein the response is a pathological complete response (pCR).

22. The method of claim 16, wherein the biological sample is a breast tumor sample.

23. The method of claim 22, wherein the breast tumor sample is a pre-treatment breast tumor sample.

24. The method of claim 16, wherein the breast cancer is HER2-negative breast cancer.

25. A method of determining whether to treat a breast cancer patient with a chemotherapy comprising the steps of:
(i) carrying out the method of claim 16 to obtain patient specific data,
(ii) determining whether to treat the breast cancer patient with a chemotherapy based on comparing the patient-specific data with at least one reference criterion, and
(iii) if the patient-specific data meets the at least one reference criterion recommending treatment of the patient with a chemotherapy.

26. The method of claim 25, wherein the chemotherapy comprises the administration of a taxane.

27. The method of claim 26, wherein the taxane is paclitaxel or docetaxel.

28. The method of claim 25, wherein the breast cancer is HER2-negative breast cancer.

29. A kit for predicting the response of a breast cancer patient to a chemotherapy comprising means for determining the level of at least two biomarkers in a biological sample of a breast cancer patient, wherein the at least two biomarkers are selected from three groups, the at least two biomarkers belonging to different groups and differ from each other, wherein the three groups comprise a first group, a second group, and a third group, wherein
the first group comprises SCUBE2, CA12, and ANXA9,
the second group comprises ELF5, ROPN1, ROPN1B, SOX10, TMEM158, FAM171A1, and SFRP1, and
the third group comprises NFIB and SFRP1.
